(19) United States
(12) Patent Application Publication
(10) Pub. No.: US 2020/0199206 A1
(43) Pub. Date: Jun. 25, 2020

(54) BACTERIAL TOXIN-NEUTRALIZING NANOBODIES AND METHODS OF MAKING AND USING THE SAME

(71) Applicant: AC Thera, LLC, Wake Forest, NC (US)

(72) Inventor: Alfred E. Corey, Wake Forest, NC (US)

(21) Appl. No.: 16/210,080

(22) Filed: Dec. 5, 2018

Related U.S. Application Data
(60) Provisional application No. 62/596,144, filed on Dec. 8, 2017.

(51) Int. Cl. C07K 16/12; C07K 16/00

(52) U.S. Cl. CPC C07K 16/1278; C07K 16/005; C07K 2317/569; C07K 2317/24; C07K 2317/22; C07K 2317/76

(57) ABSTRACT

The presently disclosed subject matter is generally directed to novel nanobodies that can be used to neutralize bacterial toxins in a subject affected with harmful bacteria, such as bacteria associated with anthrax exposure and toxic shock syndrome. The disclosed nanobodies may be capable of binding to antigenic sites that are functionally invisible to larger antibody proteins. Further, the low molecular weight and compact size of the disclosed nanobodies confer thermostability characteristics, such that they are stable in extreme pH conditions and when exposed to proteolytic digestion. Most importantly, the disclosed nanobodies can readily move from the circulatory system into tissues to disrupt the disease process.

### CROSS-REFERENCE TO RELATED APPLICATIONS

This application claims priority to U.S. Provisional Patent Application No. 62/596,144, filed Dec. 8, 2017, the entire content of which is hereby incorporated by reference.

### TECHNICAL FIELD

The presently disclosed subject matter relates generally to immunotherapeutics. More specifically, the presently disclosed subject matter relates to nanobodies that can neutralize bacterial toxins, and methods for using nanobodies to treat subjects exposed to such toxins.

### BACKGROUND

Some disease-causing bacteria produce and release toxins, and these toxins can have a significant role in morbidity and mortality. Although antibiotics can eradicate the infection associated with the bacteria, they cannot neutralize the bacterial toxins released prior to eradication of the infection. Hence, the bacterial toxins can cause damage to host tissues despite successful antibiotic therapy. The need for anti-toxin therapies was particularly exemplified by the 2001 anthrax attacks in the United States. In those attacks, there were 11 documented cases of inhalational anthrax. Despite successful eradication of the infection by the antibiotics in the 5 fatal cases, the disease continued to progress (likely due to the presence of anthrax toxins) and eventually led to the death of the individual (Jernigan J. A., Stephens D. S., Ashford D. A., et al. “Bioterrorism-related Inhalational Anthrax: The First 10 Cases Reported in the United States.” Emerg Infect Dis 7 (2001):933-44). Toxic shock syndrome represents another example of a condition where bacterial toxins have been shown to adversely affect an individual. The pyrogenic exotoxins of Group A Streptococci and Staphylococcus aureus can cause an aberrant proliferation of specific T-cell subsets, which leads to significantly increased production of cytokines that cause the shock-like symptoms (Brosnahan A. J. and Schlievert P. M. “Gram Positive Bacterial Superantigen Outside-In Signaling Causes Toxic Shock Syndrome.” FEBS J. 278 (2011): 4649-67). Toxic shock syndrome has a high mortality (estimated ˜20,000 cases annually with a 10% mortality rate (Weiss K. A. and Layerdiere M. “Group A Streptococcus Invasive Infections: a Review”. Can. J. Surg. 40 (1997): 18-25). It would therefore be beneficial to provide a treatment that neutralizes bacterial toxins in a subject affected with harmful bacteria.

### SUMMARY

[I will update this section once the claims are finalized.]

### BRIEF DESCRIPTION OF THE DRAWINGS

The previous summary and the following detailed descriptions are to be read in view of the drawings, which illustrate some (but not all) embodiments of the presently disclosed subject matter.

FIG. 1a is a representation of a human immunoglobulin G antibody.

FIG. 1b is a representation of a camelid single domain antibody that comprises variable heavy chain domains and constant heavy chain domains only.

FIG. 1c is a representation of a shark single domain antibody that comprises variable heavy chain domains and constant heavy chain domains only.

FIG. 1d is a representation of a nanobody that comprises only the variable heavy chain (V_{H}) from the antibodies shown in FIG. 1a, 1b, or 1 c.

FIG. 2a is a schematic illustrating the mechanism of action for internalization of edema factor (EF) and lethal factor (LF) resulting in toxic effects on a cell.

FIG. 2b is a schematic illustrating the blockade of toxic effects in cells by anti-PA antibodies preventing the internalization of LF and EF.

FIG. 2c is a schematic illustrating anti-LF and anti-EF nanobody neutralization of extracellular LF and EF.

FIG. 2d is a schematic illustrating nanobody neutralization of intracellular EF and LF toxins.

FIG. 3a is a schematic illustrating the mechanism of action of Staphylococcus aureus-triggered toxic shock syndrome.

FIG. 3b is a schematic illustrating the mechanism of action for toxin neutralization by a nanobody which prevents progression to toxic shock syndrome.

FIG. 4 is a schematic illustrating a method of phage display selection, phages in E. coli, and analysis in accordance with some embodiments of the presently disclosed subject matter.

FIG. 5 is a Western blot of biotinylated proteins fixed on streptavidin magnetic beads.

FIG. 6 is a schematic illustrating antibody phage display selection in accordance with some embodiments of the presently disclosed subject matter.

FIG. 7 is a bar graph illustrating LF binding during 3 rounds of phage display selection by the output/input ratio.

FIG. 8 is a bar graph illustrating LF binding results via ELISA assay for 3 rounds of phage display selection.

FIG. 9 is a schematic illustrating one embodiment of a non-adsorbed phase ELISA assay.

FIGS. 10a-10f are bar graphs illustrating non-adsorbed ELISA assay results for clones 1-25.

FIGS. 11a-11f are bar graphs illustrating non-adsorbed V_{H}H ELSIA assay results for clones 1-25.

FIG. 12 is an alignment of 10 selected clones to a reference nanobody with CDR region replaced by “x.”

FIG. 13 is a representation of a map of V_{F}H ref pHEN2.

FIG. 14 is a Western blot of biotinylated proteins fixed on streptavidin magnetic beads.

FIG. 15 is a bar graph illustrating EF binding during 3 rounds of phage display selection by the output/input ratio.

FIG. 16 is a bar graph illustrating EF binding results via ELISA assay for 3 rounds of phage display selection.

FIGS. 17a-17g are bar graphs illustrating non-adsorbed ELISA assay results for clones 26-66.

FIGS. 18a-18g are bar graphs illustrating non-adsorbed ELISA IPTG assay results for clones 26-66.

FIG. 19 is an alignment of 10 clones selected from clones 26-66 compared to a reference nanobody with CFR region replaced by “x.”

FIG. 20 is a schematic illustrating phage display selection, GAP repair, and yeast clone production in accordance with some embodiments of the presently disclosed subject matter.

FIG. 21 is a Western blot of biotinylated proteins fixed on streptavidin magnetic beads.

FIG. 22 is a bar graph illustrating TSST1 binding during 3 rounds of phage display selection by the output/input ratio.

FIG. 23 is a bar graph illustrating TSST1 binding results via ELISA assay for 3 rounds of phage display selection.

FIG. 24 is a bar graph illustrating V_{H}Hs control for ELISA assay.

FIG. 25 is an alignment of 10 clones selected from clones 67-94 compared to a reference nanobody with CFR region replaced by “x.”

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a nanobody” can include a plurality of such nanobodies, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the scope of the presently disclosed subject matter.

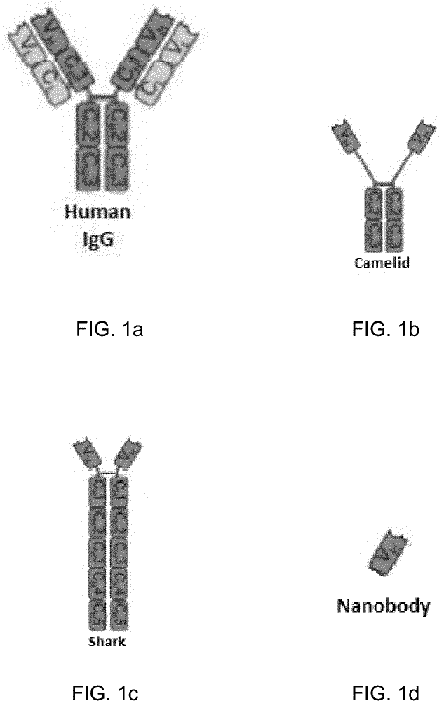

The presently disclosed subject matter is generally directed to novel nanobodies that can be used to neutralize bacterial toxins in a subject infected with harmful bacteria. As used herein, the term “nanobody” refers to a single-domain antibody fragment that comprises the unique structural and functional properties of naturally-occurring heavy chain only antibodies. Thus, nanobodies are antibody-derived therapeutically active proteins. Like whole antibodies, nanobodies are able to bind selectively to a specific antigen. FIG. 1a illustrates human immunoglobulin G (IgG) antibody that includes a light chain with two domains, the variable light chain domain (V_{L}) and the constant light chain domain (C_{L}). IgG antibody further comprises a heavy chain with four domains, one variable heavy chain domain (V_{H}) and three constant heavy chain domains (C_{H}1, C_{H}2, and C_{H}3).

In some embodiments, the disclosed nanobodies can be camelid-derived (i.e., obtained from members of the camelid family) that specifically bind to an antigen or a fragment thereof. Suitable members of the camelid family can include (but are not limited to) Camelus bactrianus and Camelus dromaderius, as well as llama species (Vicugna pacos, Lama glama and Vicugna vicugna). Advantageously, various IgG antibodies from the camelid family lack light chains as illustrated in FIG. 1b, and are thus structurally distinct from the typical four chain quaternary structure having two heavy and two light chains. The small single variable region (identified as V_{H}) of the camelid domain can be obtained by genetic engineering to yield a nanobody having high affinity for a desired target. Such methods are well known in the art. Nanobodies engineered from heavy-chain antibodies found in camelids are termed “V_{H}H fragments.”

Cartilaginous fishes also have heavy-chain antibodies (IgNAR, “immunoglobulin new antigen receptor”), from which single-domain antibodies (called VNAR) can be obtained. Suitable cartilaginous fishes can include (but are not limited to) sharks. Particularly, certain species of shark produce functional antigen-binding heavy chain antibodies naturally devoid of the light chain. As shown in FIG. 1c, the shark antibody comprises five constant domains (C_{H}1, C_{H}2, C_{H}3, C_{H}4, C_{H}5) and one variable domain (V_{H}). As with camelid-derived nanobodies, the variable domain (V_{H}) of the cartilaginous fish antibody can be isolated using genetic engineering to yield a nanobody having high affinity for a target.

As an alternative, the dimeric variable domains from common human or murine IgG can be split into monomers (V_{H} and V_{L} fragments). Such methods are well known to those of ordinary skill in the art.

As shown in FIG. 1d, the illustrated nanobody comprises only the variable heavy chain (V_{H}) of the antibody of FIG. 1a, FIG. 1b, or FIG. 1c.

In some embodiments, the disclosed nanobodies (e.g., camelid-derived or shark-derived) can be humanized. As used herein, the term “humanized nanobody” refers to a nanobody wherein one or more amino acid residues of the naturally occurring amino acid sequence of the antibody domain has been replaced by one or more amino acid residues that occur at the corresponding position(s) in a VH domain from a conventional chain human antibody. Methods of humanizing single domain antibodies are well known in the art. Typically, the humanizing substitutions can be selected such that the resulting humanized nanobodies retain favorable properties, such as binding specificity. Those skilled in the art would be able to determine and select suitable humanizing substitutions and/or suitable combinations of humanizing substitutions.

Advantageously, nanobodies have a molecular weight that is approximately 1/10 that of a human IgG molecule. As a result, nanobodies exhibit desirable characteristics while maintaining the target specificity of antibodies (Harmsen M. M. and De Haard H. J., “Properties, Production, and Applications of Camelid Single-Domain Antibody Fragments”. Appl Microbiol Biotechnol 77 (2007): 13-22). For example, one benefit of the small size of the disclosed nanobodies is the ability to bind to antigenic sites that are functionally invisible to larger antibody proteins. Further, the low molecular weight and compact size of the disclosed nanobodies confer thermostability characteristics, such that they are stable in extreme pH conditions (e.g., pH 1-3 and/or 11-14) and when exposed to proteolytic digestion. Most importantly, the disclosed nanobodies can readily move from the circulatory system into tissues. Table 1 summarizes several key differences between nanobodies and antibodies.

TABLE 1
Comparison of Antibody and Nanobody Characteristics^{a}

| | Antibody | Nanobody |
|---|---|---|
| Molecular Weight | 150-160 kDa | 12-15 kDa |
| Specificity | Similar | Similar |
| Tissue Distribution | Limited | Better |
| Intracellular Distribution | Poor | Good |
| Distribution Across Blood- | Negligible | Some |
| Brain Barrier | | |
| Ability to Target Enzyme | Limited | Better |
| Active Sites | | |
| Elimination Half-Life | About 20 days^{b} | About 2 hours |
| Subject to Renal | No | Yes |
| Elimination | | |
| Heat Resistance | Limited | Better |
| Stability to Urea/Detergent | Limited | Better |
| Resistance to Gastric | Limited | Better^{c} |
| Acid/Proteases | | |
| Complement Fixation | Yes | No |
| Sources | Human, murine, most | Camelids, |
| | mammals | Cartilaginous |
| | | fishes |
| Ability to be Fully | Yes | Yes |
| Humanized | | |
| Production Systems | Usually mammalian due | Yeast or bacteria |
| | to post-expression | can be used |
| | modifications e.g., | |
| | glycosylation) | |
| Immunogenicity Potential | Variable | Low^{d} |

^{a}Based on: Harmsen M M and De Haard H J., 2007.
^{b}For a typical human antibody; half-lives may be shorter for chimeric antibodies or antibodies of nonhuman origin.
^{c}Strategies have been reported for improving resistance (Harmsen M M and De Haard H J., 2007)
^{d}Based on small size, stable behavior, rapid clearance from blood, and a sequence sharing a high degree of identity with human V_{H}; strategies exist for humanization.

In some embodiments, the presently disclosed subject matter includes isolated nanobodies that specifically bind to B. anthracis toxins (such as lethal factor (LF) and edema factor (EF)) and S. aureus toxins (such as toxic shock syndrome toxin 1 (TSST-1)). However, it should be appreciated that the presently disclosed subject matter is not limited and can include nanobodies that neutralize a wide variety of bacterial toxins.

The most severe form of anthrax infection occurs following inhalation of B. anthracis spores that germinate within macrophages as they travel to the draining mediastinal lymph nodes in a subject. Although the bacterial replication (bacteremia) can be controlled by administration of appropriate antibiotics, the bacterial toxins exert deleterious effects on the cells within the body. As a result, substantial pathology and high mortality occurs in infected individuals. Because they have no direct effect on the toxins, antibiotics are unable to treat the effects of the toxins.

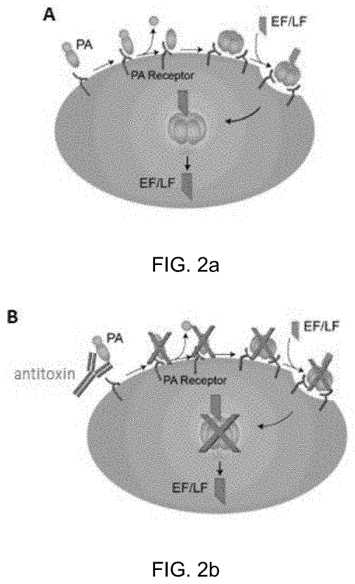

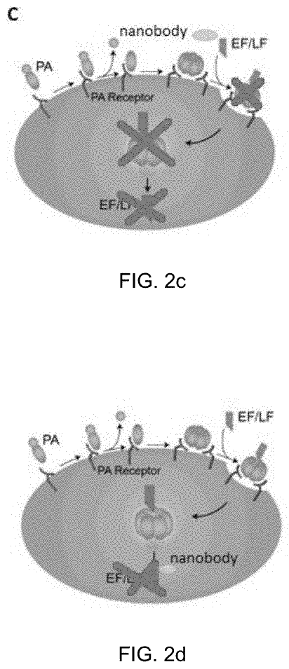

B. anthracis produces 3 toxins, which include a binding moiety, protective antigen (PA), and 2 enzymatic moieties, edema factor (EF) and lethal factor (LF) (Ascenzi P., Visca P., Ippolito G., et al. “Anthrax toxin: A Tripartite Lethal Combination.” FEBS Letters 531 (2002): 384-8; Inglesby T. V., O'Toole T., Henderson D. A., et al. “Anthrax as a Biological Weapon, 2002: Updated Recommendations for Management.” JAMA 287 (2002): 2236-52). The mechanism of action of B. anthracis is illustrated in the schematic of FIG. 2a. Particularly, the bacterial PA antigen first binds to its cell surface receptor and is cleaved by a membrane-bound furin-like protease leaving a 63 kDa fragment bound to the cell. The bound antigen fragment multimerizes into a heptameric barrel structure and exposes a site to which edema factor (EF) and lethal factor (LF) bind with high affinity. Internalization of the complex by receptor-mediated endocytosis is followed by the formation of a membrane-spanning pore. The bound EF and LF proteins are then translocated through the pore to the cytosol of the cell to exert a toxic effect.

FIG. 2b is a schematic illustrating immunity to anthrax infection conferred by stimulating the body to produce antitoxin antibody (Friedlander A. M., Welkos S. L., Ivins B. E. “Anthrax Vaccines”. Curr. Top. Microbiol. Immunol. 271 (2002): 33-60) or by passive immunization, e.g. administration of an anti-PA antibody. As shown, the antitoxin antibodies prevent PA binding to cell surface receptors, thereby preventing internalization of LF and EF. However, the toxic effects in cells that have internalized LF and EF are still present, leading to cell death. To this end, multiple published studies have shown that antibiotics or anti-PA antibodies can be 100% effective in the treatment of inhalational anthrax if administered early enough after exposure to B. anthracis spores. However, when treatment is delayed until after the onset of symptoms, not all treated subjects survive (Hou A. W., Morrill A. M. “Obiltoxaximab: Adding to the Treatment Arsenal for Bacillus anthracis Infection.” Ann. Pharmacother. 51 (2017): 908-13; Huang, E., Pillai, S. K., Bower W. A., et al. “Antitoxin Treatment of Inhalation Anthrax: A Systematic Review.” Health Secur. 13 (2015): 365-77; Rubinson L., Corey A., Hanfling D. “Estimation of Time Period for Effective Human Inhalational Anthrax Treatment Including Antitoxin Therapy”. PLOS. Curr. Outbreaks. 1: (2017)).

Two monoclonal anti-PA antibodies (Raxibacumab and Obiltoxaximab) that afford passive immunization have been approved by the Food and Drug Administration (Tsai C-W, and Morris S. “Approval of Raxibacumab for the Treatment of Inhalation Anthrax Under the U.S. Food and Drug Administration ‘Animal Rule.’” Front. Microbiol. 6 (2015): 1320; Greig S. L. “Obiltoxaximab: First Global Approval.” Drugs. 76 (2016): 823-30). Anti-PA bispecific V_{H}H-based agent (2 linked anti-PA nanobodies) have also been reported as providing prophylactic protection from anthrax (Moayeri M., Tremblay J. M., Debatis M., et al. “Adenoviral Expression of a Bispecific V_{H}H-Based Neutralizing Agent that Targets Protective Antigen Provides Prophylactic Protection from Anthrax in Mice”. Clin. Vaccine Immunol. 23 (2016): 213-8). The latter provides support for the assertion that using nanobodies to neutralize LF/EF should have a therapeutic benefit.

The mortality observed with delayed post-exposure treatment is believed to result from the deleterious effects of intracellular LF and EF, which antibiotics and anti-PA antibodies do not neutralize. Thus, anti-LF and anti-EF nanobodies have the potential to protect against extracellular toxins, as shown in the schematic of FIG. 2c. Particularly, anti-LF and anti-EF nanobodies neutralize extracellular EF and LF, thereby preventing internalization of LF and EF. As a result, the cells are spared any toxic effects. As illustrated in the schematic of FIG. 2d, in cells that have already internalized bacterial toxins, nanobodies enter the cytosol to neutralize intracellular toxins, thereby ceasing the deleterious effects that lead to cell death.

Thus, nanobodies offer an advantage over existing therapies and hold the potential to prevent the mortality associated with delayed post exposure treatment of bacterial infections, such as inhalational anthrax. However, use of the disclosed nanobodies to treat, prevent, and/or ameliorate bacterial infections is not limited to B. anthracis infections. For example, the disclosed nanobodies are also believed to be beneficial in the treatment of toxic shock syndrome. Toxic shock syndrome is primarily caused by Streptococcus pyogenes or S. aureus (Low, D. E. “Toxic Shock Syndrome: Major Advances in Pathogenesis, but not Treatment.” Critical Care Clinics. 29 (2013): 651-75). Antibiotics are the first-line treatment for toxic shock syndrome, but the mortality rate remains unacceptably high (Lappin E., Ferguson A. J. “Gram-Positive Toxic Shock Syndromes.” Lancet Infect. Dis. 9 (2009): 281-90). Bacterial toxin-mediated activation of the inflammatory cascade is believed to be the primal event in toxic shock syndrome pathogenesis.

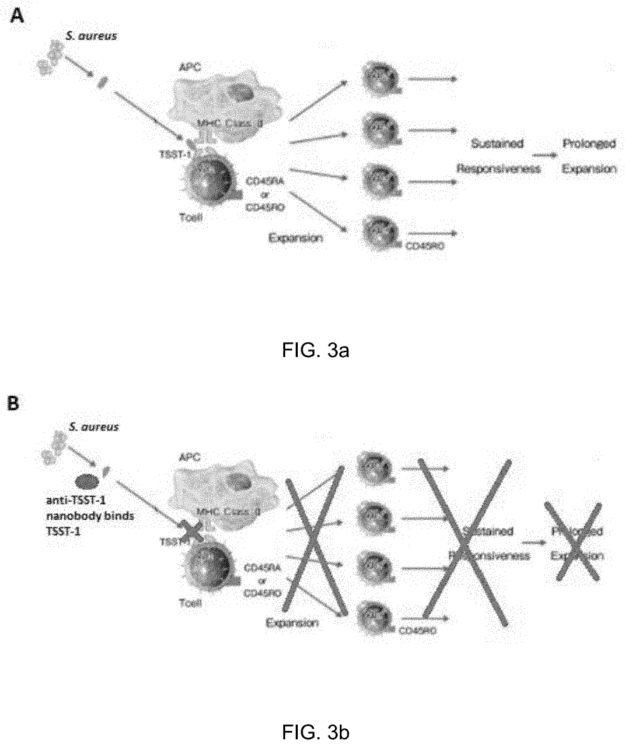

The toxic shock syndrome toxin 1 (TSST-1) of S. aureus is considered to have a pivotal role in the pathogenesis of toxic shock syndrome (Schlievert P. M., Osterholm M. T., Kelly J. A., et al. “Toxin and Enzyme Characterization of S. aureus Isolated from Patients with and without Toxic Shock Syndrome.” Ann. Intern. Med. 96 (1982): 937-40). The schematic of FIG. 3a illustrates the role of TSST-1 as instigator of the cascade that manifests as toxic shock syndrome. Specifically, S. aureus releases TSST-1, which binds to major histocompatibility complex (MHC) Class II receptors on antigen presenting cells (APC) and receptors on T cells, resulting in proliferation of T cells and subsequent release of cytokines. The symptoms of toxic shock syndrome develop as a result of the proliferation and cytokine release. FIG. 3b illustrates a schematic wherein an anti-TSST-1 nanobody blocks the instigator action by preventing TSST-1 from binding to cell surface receptors, preventing the proliferation/cytokine release cascade.

TSST-1 is just one of many superantigens (Proft T., Fraser J. D. “Bacterial Superantigens.” Clin. Exp. Immunol. 133 (2003): 299-306; Dinges M. M., Orwin P. M., Schlievert P. M. “Exotoxins of Staphylococcus aureus.” Clin. Microbiol. Rev. 13 (2000): 16-34). TSST-1 from S. aureus shares similar biological activity with the staphylococcal enterotoxins (SE) and streptococcal pyrogenic exotoxins (SPE), although the amino acid sequence of TSST-1 differs from these two classes of toxins (Proft and Fraser, 2003). More than 20 SE have been identified (Pinchuk I. V., Beswick E. J., Reyes V. E. “Staphylococcal Enterotoxins.” Toxins (Basel) 2 (2010): 2177-97), and at least 11 SPE are known (Proft and Fraser, 2003). Given the relatively unique structure of TSST-1 and its primary role in the development of toxic shock syndrome, it is believed that anti-TSST-1 nanobodies can be used to help prevent development of toxic shock syndrome.

However, it should be appreciated that neutralization of other superantigens can also be desirable based on the known synergistic effects of TSST-1 with the SE/SPE family of toxins (Smith R. J., Schlievert P. M., Himelright I. M., et al. “Dual Infections with Staphylococcus aureus and Streptococcus pyogenes Causing Toxic Shock Syndrome. Possible Synergistic Effects of Toxic Shock Syndrome Toxin 1 and Streptococcal pyrogenic Exotoxin C.” Diagn. Microbiol. Infect. Dis. 19 (1994): 245-7; Stevens D. L., Tanner M. H., Winship J., et al. “Severe Group A Streptococcal infections Associated with a Toxic Shock-like Syndrome and Scarlet Fever Toxin A.” N. Engl. J. Med. 321 (1989): 1-7). Gram negative bacterial endotoxins and the pyrogenic toxins can work synergistically to produce septic shock (Bannan J., Visvanathan K., Zabriskie J. B. “Structure and Function of Streptococcal and Staphylococcal Superantigens in Septic Shock.” Infect. Dis. Clin. North Am. 13 (1999): 387-96).

In some embodiments, the presently disclosed subject matter relates generally to polypeptides that comprise a V_{H}H of a camelid heavy chain nanobody or a VNAR domain of a cartilaginous fish heavy chain antibody. In some embodiments, the polypeptides bind to a target selected from B. anthracis edema factor (EF), B. anthracis lethal factor (LF), and S. aureus toxic shock syndrome toxin/(TSST-1) to thereby neutralize the target.

The disclosed nanobodies are thus capable of selectively binding to EF, LF, and/or TSST-1. In some embodiments, the disclosed nanobodies are specific for EF, LF, and/or TSST-1 (e.g., do not bind to any other molecules). In some embodiments, the presently disclosed subject matter is directed to a nanobody directed against LF comprising the amino acid sequence of one or more of the amino acid sequences of SEQ ID NO: 1-10 and/or the nucleotide sequences set forth in SEQ ID NO: 31-40. In some embodiments, the presently disclosed subject matter is directed to a nanobody directed against EF comprising the amino acid sequence of one or more of the amino acid sequences of SEQ ID NO: 11-20 and/or the nucleotide sequences set forth in SEQ ID Nos: 41-50. In some embodiments, the presently disclosed subject matter is directed to a nanobody directed against TSST-1 comprising the amino acid sequence of one or more of the amino acid sequences of SEQ ID NO: 21-30 and/or the nucleotide sequences set forth in SEQ ID NO: 51-60.

Thus, in some embodiments, the disclosed nanobody can comprise the amino acid sequence of one or more of SEQ ID NOs: 1-30. In some embodiments, the disclosed nanobody can have an amino acid sequence with a sequence identity of at least about 60% with the amino acid sequence of one or more of SEQ ID Nos: 1-30. Thus, the presently disclosed subject matter can include a nanobody comprising an amino acid sequence with a sequence identity of at least about 60, 65, 70, 75, 80, 85, 90, 91, 92, 93, 94, 95, 96, 97, 98, 99, or 99.9% with one or more of SEQ ID NOs: 1-30. Alternatively or in addition, the nanobody can comprise a functional fragment of the amino acid sequence of one or more of SEQ ID NOs:1-30.

In some embodiments, the disclosed nanobody can comprise the nucleotide sequence of one or more of SEQ ID NOs: 31-60. In some embodiments, the disclosed nanobody can have a nucleotide sequence with a sequence identity of at least about 60% with the nucleotide sequence of one or more of SEQ ID Nos: 31-60. Thus, the presently disclosed subject matter can include a nanobody comprising a nucleotide sequence with a sequence identity of at least about 60, 65, 70, 75, 80, 85, 90, 91, 92, 93, 94, 95, 96, 97, 98, 99, or 99.9% with one or more of SEQ ID NOs: 31-60. Alternatively or in addition, the nanobody can comprise a functional fragment of the nucleotide sequence of one or more of SEQ ID NOs:31-60.

The term “sequence identity” in the context of nucleic acid or polypeptide sequences refers to the nucleic acid bases or amino acid residues in the two sequences that are the same when aligned for maximum correspondence over a specified comparison window. Thus, “percentage of sequence identity” refers to the valued determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include (but are not limited to) 50%, 55%, 60%, 65%, 70%, 75%, 80%, 85%, 90%, or 95%, or any integer percentage from 55% to 100%.

Optimal alignment of sequences for aligning a comparison window are well known to those skilled in the art and can be conducted by tools such as the local homology algorithm of Smith and Waterman, the homology alignment algorithm of Needleman and Wunsch, the search for similarity method of Pearson and Lipman, and/or by computerized implementations of these algorithms such as GAP, BESTFIT, FASTA, and TFASTA available as part of the GCG® Wisconsin Package® (Accelrys Inc., Burlington, Mass.).

The disclosed nanobodies can be modified, e.g., by the covalent attachment of any type of molecule provided that the covalent attachment does not prevent the nanobody from binding to its antigen. Examples of suitable modifications can include (but are not limited to) glycosylation, acetylation, pegylation, phosphorylation, amidation, and the like. In some embodiments, the nanobodies can be derivatized by known protecting/blocking groups, proteolytic cleavage, linkage to a cellular ligand or other proteins, and the like. In some embodiments, the nanobodies can comprise post-translational moieties that improve upon nanobody activity or stability, such as (but not limited to) sulfur, methyl, carbohydrate, phosphorus, and the like.

It should be appreciated that functionally conservative variants of the disclosed nanobodies are also included within the scope of the presently disclosed subject matter. The term “functionally conservative variant” refers to variants in which a given amino acid is substituted without altering the global conformation and the function of the nanobody, including a replacement of an amino acid with another having similar properties (for example polarity, hydrogen bond potential, acidity, basicity, hydrophobicity, presence of an aromatic group, etc). The amino acids having similar properties are well known to one skilled in the art. The functionally conservative variants preserve their capability of binding LF, EF, and/or TSST-1. In some embodiments, functionally conservative variants can have a bond affinity with LF, EF, and/or TSST-1 equal or increased relatively to the corresponding nanobody.

By knowing the nucleotide and amino acid sequences of a nanobody of interest, one skilled in the art would be capable of producing the nanobodies according to the presently disclosed subject matter by conventional techniques. For example, the nanobodies can be synthesized by using a well-known synthesis method in a solid phase (Merrifield (19962) Proc. Soc. Ex. Boil. 21:412; Merrifield (1963) J. Am. Chem. Soc. 85:2149; Tam et al. (1983) J. Am. Chem. Soc. 105:6442), by using a commercially available peptide synthesis apparatus, (such as the one made by Applied Biosystems, Foster City, Calif.) and by following the instructions of the manufacturer. Alternatively, the nanobodies can be synthesized with recombinant DNA techniques well-known in the art (e.g., Maniatis et al. (1982) Molecular Cloning: a laboratory manual, Cold Spring Harbor Laboratories, NY, 51-54 and 412-430). For example, the nanobodies can be obtained as DNA expression products after incorporating DNA sequences coding for the polypeptide of interest in expression vectors and introducing these vectors in suitable prokaryotic or eukaryotic hosts that will express the polypeptide of interest, from which they may then be isolated by using techniques well known to one skilled in the art. A nanobody comprising or consisting of the sequence of amino acids selected from SEQ ID NOs: 1-30 and/or the sequence of nucleotides selected from SEQ ID NO: 31-60 is also included within the scope of the presently disclosed subject matter.

The disclosed nanobodies can generally be used to treat, prevent, and/or ameliorate a condition characterized by the presence of one or more bacterial toxins. For example, in some embodiments, the disclosed nanobodies can inhibit or prevent binding of bacterial toxins to their receptors. Alternatively or in addition, the disclosed nanobodies can interrupt a bacterial toxin pathway to prevent translocation of the bacterial toxins into a cell. Further, the disclosed nanobodies can modulate the signaling, enzymatic activity, and/or biological mechanisms mediated by one or more bacterial toxins. Thus, the disclosed nanobodies can be used for the prevention and treatment of bacterial toxin-related conditions, such as anthrax exposure and/or toxic shock syndrome.

In some embodiments, the disclosed nanobodies can bind to a bacterial toxin or fragment thereof, such as (but not limited to) B. anthracis edema factor (EF), B. anthracis lethal factor (LF), and/or S. aureus toxic shock syndrome toxin1 (TSST-1) to neutralize the effects of the toxin. As used herein, the term “neutralize” refers to the ability of a nanobody to inhibit activity of an antigen to which the nanobody binds. Thus, in some embodiments, the disclosed nanobodies can be anti-LF, anti-EF, or anti-TSST1 nanobodies, and are specific for LF, EF, and TSST-1, respectively. As a result, the deleterious conditions caused by the bacterial toxins are ameliorated or eliminated. The nanobodies can neutralize the LF, EF, and/or TSST-1 such that the toxins are unable to cause physiological damage to the cell.

The presently disclosed subject matter therefore provides for a pharmaceutical composition comprising any of the nanobodies described herein and a pharmaceutically acceptable carrier. Pharmaceutical compositions can be provided as substantially free from any etiologic agents, infectious agents (particularly those infectious for the subject to which the composition is to be administered, e.g., a human), human blood components, and the like. In some embodiments, the nanobodies and associated pharmaceutical compositions are substantially free from any contaminants normally found in a mammalian or bacterial cell extract or preparations.

When the disclosed nanobodies are humanized, they are significantly less immunogenic and more therapeutically effective and useful when administered to human patients than non-humanized nanobodies.

In some embodiments, the presently disclosed subject matter comprises a pharmaceutical composition that includes the disclosed nanobodies. The disclosed nanobodies can be administered to a subject as a pharmaceutical preparation. Particularly, the pharmaceutical preparation can comprise at least one isolated nanobody polypeptide and optionally at least one physiologically acceptable carrier. The term “carrier” refers to a non-toxic material that does not interfere with the effectiveness of the biological activity of the nanobodies, such as (but not limited to) sterile water, aqueous buffers (e.g., phosphate-buffered saline, Ringer's solutions, dextrose solution, Hank's solution), buffered saline, ethanol, polyol, glycerol, alcohol (e.g., ethanol), and/or glycol (e.g., propylene glycol). The term “physiologically acceptable” refers to a non-toxic material that is compatible with a biological system such as a cell, cell culture, tissue, and/or organism. As would be known to those of ordinary skill in the art, the characteristics of the carrier will depend on the route of administration. Physiologically and pharmaceutically acceptable carriers can include (but are not limited to) diluents, fillers, salts, buffers, stabilizers, solubilizers, and other materials that are well known in the art.

The pharmaceutical preparations can be formulated and administered in any suitable manner known or used in the art. See, for example, Remington's Pharmaceutical Sciences, 18^{th} Ed., Mack Publishing Company, USA (1990) or Remington, the Science and Practice of Pharmacy, 21th Edition, Lippincott Williams and Wilkins (2005), the entire contents of which are hereby incorporated by reference. For example, the pharmaceutical preparations can be formulated for parenteral administration (e.g., intravenous, intraperitoneal, subcutaneous, intramuscular, intraluminal, intra-arterial or intrathecal administration), oral administration, and/or for topical administration (e.g. transdermal or intradermal).

In addition to the carrier, the disclosed formulation can optionally comprise one or more additives in an amount of from about 0.01-5 weight percent. The additives can include one or more binders, thickeners, softeners, dispersions, emulsifiers, preservatives, lubricants, enzymes, sweeteners, perfumes, pigments, pH adjusting agents, fillers, humectants, absorption accelerators, wetting agents, absorbents, and the like. Such additives are well known to those of ordinary skill in the art. In some embodiments, the additives and/or carriers are inert and/or non-reactive with the nanobodies.

The pharmaceutical composition can further optionally comprise one or more additional therapeutic agents. Any suitable therapeutic agent can be used, such as (but not limited to) an antibiotic (e.g., ciprofloxacin, doxycycline, chloramphenicol, clindamycin, tetracycline, rifampin, and/or vancomycin), a vaccine, an antibody, and/or a second nanobody. In some embodiments, the compositions include a combination of multiple (e.g., two or more) anti-EF, anti-LF, and/or anti-TSST-1 nanobodies, or antigen-binding fragments thereof.

The compositions can be administered to the subject using any known administration route. For example, in some embodiments, the nanobodies can be administered orally (through the mouth), sublingually (under the tongue), topically (absorbed through the skin), nasally (through the nasal passages), intravenously (injected into a vein), subcutaneously (under the skin), parentally (injection) and/or by inhalation (by breathing through the airway). The nanobodies can therefore be formulated as capsules, cachets, pills, tablets, lozenges, powders, granules, inhalant, or as a solution or a suspension in an aqueous or non-aqueous liquid. Depending on the route of administration, the nanobody can be coated in a material to protect the compound from the action of acids and other natural conditions that may inactivate the compound.

The nanobody formulations can conveniently be presented in unit dosage form and can be prepared by any methods well known in the art of pharmacy. The amount of active ingredient (e.g., nanobody) that can be combined with a carrier material to produce a single dosage form will generally be an amount that produces a therapeutic effect. In some embodiments, the formulation can comprise about 1-99 weight percent active ingredient (e.g., 1-99, 2-90, 5-80, 10-70, 25-60, 30-50 weight percent) and about 99-1 weight percent carrier (e.g., 99-1, 98-10, 95-20, 90-30, 75-40, or 70-50 weight percent).

Actual dosage levels of the nanobodies in the disclosed pharmaceutical compositions can be varied to obtain an amount of the active ingredient that is effective to achieve the desired therapeutic response for a particular patient, composition, and mode of administration without being toxic to the patient. The selected dosage level will depend upon a variety of factors including the activity of the particular compositions, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the nanobody employed in the pharmaceutical composition at levels lower than that required in order to achieve the desired therapeutic effect and gradually increase the dosage until the desired effect is achieved. In general, a suitable daily dose of a composition will be that amount of the compound which is the lowest dose effective to produce a therapeutic effect. Such an effective dose will generally depend upon the factors described above.

The term “therapeutically effective” in reference to dose of a nanobody refers to an amount that reduces a sign or a symptom associated with anthrax infection, anthrax toxin toxicity, a sign or a symptom associated with toxic shock syndrome infection, and/or toxic shock syndrome toxicity by at least about 20%, about 40%, about 60%, about 80%, or by complete reduction relative to untreated subjects. In some embodiments, a therapeutically effective amount of a nanobody is an amount sufficient to prevent death in a subject exposed to anthrax and/or toxic shock syndrome. The ability of a nanobody to reduce signs and/or symptoms (including preventing death) associated with anthrax and/or toxic shock syndrome infection can be evaluated in an animal model system predictive of efficacy of the nanobody in treating human anthrax and/or toxic shock syndrome infection.

Alternatively, a therapeutically effective amount of a nanobody can be evaluated by examining the ability of the nanobody to neutralize an anthrax toxin or toxic shock syndrome toxin in vitro in a toxin neutralization assay. Such assays are well known in the art. One of ordinary skill in the art would be able to determine such therapeutically effective amounts based on factors such as the subject's size, the severity of the signs and/or subject's symptoms, and/or the particular composition or route of administration selected.

The presently disclosed subject matter also provides for a method of treating or reducing the toxicity of anthrax toxin and/or toxic shock syndrome toxin. Such methods find application both in vivo and in vitro. For in vivo methods, an anti-anthrax (e.g., anti-EF or anti-LF) or anti-toxic shock syndrome (e.g., anti-TSST-1) nanobody is administered to a subject. For in vitro methods, the nanobody is contacted with a material suspected of having anthrax toxin or toxic shock syndrome toxin (e.g., as a material suspected of being contaminated with anthrax toxin) in an amount effective to provide for neutralization of toxin by the nanobody.

Any subject having or susceptible to anthrax toxin or toxic shock syndrome toxin exposure can be treated according to the methods disclosed herein. In some embodiments, the subject is infected with B. anthracis and/or S. aureus, has been exposed to anthrax or toxic shock syndrome toxin, or is at risk of infection or exposure.

In related embodiments, the disclosure provides methods for detecting the presence of B. anthracis and/or S. aureus in a sample, such as a biological sample (e.g., blood from an anthrax-infected subject). The method includes contacting a sample suspected of containing B. anthracis toxin and/or S. aureus toxin with one or more of the disclosed nanobodies (e.g., the anti-LF, anti-EF, and/or anti-TSST-1 nanobodies of SEQ ID NOs: 1-30). The contacting occurs under conditions to allow for formation of a specific nanobody-antigen complex. The presence or absence of the complex is then detected, wherein the presence of the complex indicates the presence of a nanobody-antigen complex. The presence of the nanobody-antigen complex indicates the presence of B. anthracis toxin and/or S. aureus toxin in the sample. For example, a blood or tissue sample can be removed from a subject and contacted with a nanobody under conditions which allow detection of anthrax EF and/or LF in the sample to diagnose an anthrax infection in the subject. Similarly, detection of TSST-1 in a sample can be used to diagnose toxic shock syndrome infection in the subject.

Suitable immunodetection techniques include (but are not limited to) both in vitro methods and in vivo (imaging) methods. Where the methods are in vitro, the biological sample can be any sample in which a selected antigen is present (e.g., blood samples (including whole blood, serum, etc.), tissues, whole cells (e.g., intact cells), tissue or cell extracts, as well as samples obtained from other sources (e.g., packages, letters, food products, and the like). Assays can take a wide variety of forms, such as competition, direct reaction, or sandwich type assays. Exemplary assays include Western blots; enzyme-labeled and mediated immunoassays, such as ELISAs; biotin/avidin type assays; radioimmunoassays; immunoelectrophoresis; immunoprecipitation, and the like. The reactions generally include detectable labels such as fluorescent, chemiluminescent, radioactive, enzymatic labels or dye molecules, or other methods for detecting the formation of a complex between antigen in the sample and the nanobody.

The term “subject” refers to a mammal, such as a human. However, in some embodiments, the subject can include (but is not limited to) domesticated animals (e.g., dogs, cats, and the like), farm animals (e.g., cows, pigs, sheep, horses, and the like), and laboratory animals (e.g., rats, mice, monkeys, and the like).

Therefore, the disclosed nanobodies offer an advantage over existing therapies and hold the potential to prevent the harmful effects and/or death associated with delayed post-exposure treatment of a variety of bacterial infections.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Methods

### Phage ELISA IPTG Plaque Assay

Preculture:

96 well Costar® plates (available from Corning, Inc. Corning, N.Y.) with a volume of 200 μl/well were used. Controls wells on each plate were as follows: A12-empty (control for contamination); A6-control anti 1329v4-A220 (positive control); B6-R3TF3 anti-GFP (negative control). The plates were incubated with agitation at 37° C. overnight in 2×TY media (1.6% tryptone, 1% yeast extract, 0.5% NaCl) with 2% glucose, 100 μg/ml Ampicillin. The box was kept in 30% glycerol (stock at −20° C.).

Culture:

In a new plate, the preculture was diluted 1/100 (6 μl of preculture for 600 μl of medium) in 2×TY media with 0.1% glucose and 100 μg/ml ampicillin. The plate was incubated for 90 minutes at 37° C. with agitation. Isopropyl β-D-1-thiogalactopyranoside (IPTG) was added at final concentration of 1 mM (stock: 840 mM). The plate was incubated overnight at 30° C. with agitation. The plate was spun at 3300 rpm for 10 minutes, and the supernatants were retained.

ELISA:

Coating was performed using Nunc® Maxisorp® plates for 2 hours at room temperature or overnight at 4° C. with 12.5 μl/well of Avidin at 20 μg/ml in PBS. The plates were washed with 50 μl PBS+0.1% Tween, and washed with 50 μl PBS. 12.5 μl of biotinylated antigen (5 μg/ml) in PBS (phosphate buffered saline) was loaded into each well of the plates. The plates were incubated 2 h at room temperature or overnight at 4° C. Saturation: 50 μl/well PBS 0.1% Tween 2% milk were incubated for 1 hour at room temperature or overnight at 4° C. 3 washes with 50 μl of PBS+0.1% Tween were performed and 3 washes with 50 μl PBS were performed.

The phages were pre-incubated for 20 minutes at room temperature. 48 μl of phages and 12 μl PBS+0.1% Tween+2% milk were added to each well. The phages were incubated 90 minutes at room temperature. The wells were then washed once with 50 μl of PBS+0.1% Tween and washed twice with 50 μl PBS.

12.5 μl/well Myc was added at a dilution of 1/500 in PBS+0.1% Tween+2% milk, and was incubated for 1 hour at room temperature. Each well was then washed with 50 μl of PBS+0.1% Tween and washed twice with 50 μl PBS.

12.5 μl/well secondary antibody anti-mouse HRP was added to each well at a dilution of 1/5000 and incubated for 1 hour at room temperature. The wells were washed 3× with 50 μl of PBS+0.1% Tween and 3 washes with 50 μl PBS.

Detection: TMB (transparent plates) or ECL (White plates), 25 μl/well TMB (3,3′,5,5″-Tetramethylbenzidine) was added. The reaction was stopped by adding 25 μl of sulfuric acid 2N (1 M). A spectrometer reading at DO=450 nm was then taken.

Phage ELISA Plaque Assay

Preculture:

performed as set forth above for the Phage ELISA IPTG plaque.

Culture:

In a new plate, the preculture was diluted 1/100 (6 μl of preculture for 600 μl of medium) in 2×TY media with 0.1% glucose and 100 μg/ml ampicillin. The plate was incubated for 2.5 hours at 37° C. with agitation. Phage helper was added from a stock at 6×10¹³/mL (4×10⁹/well). The plate was incubated 30 minutes at 37° C. without agitation. The plate was spun at 2500 rpm for 5 minutes, and the supernatants were removed by pouring the plate. Pellets were resuspended in 1 mL 2×TY medium+ampicillin (100 ug/mL); Kara 50 ug/mL without glucose. The plate was incubated overnight at 30° C. with agitation. The plate was re-spun for 10 minutes at 3300 rpm and the supernatants were retained.

ELISA:

Coating was performed using Nunc® Maxisorp® plates for 2 hours at room temperature or overnight at 4° C. with 12.5 μl/well of Avidin at 20 μg/ml in PBS. The plates were washed with 50 μl PBS+0.1% Tween, and washed with 50 μl PBS. 12.5 μl of biotinylated antigen (5 μg/ml) in PBS (phosphate buffered saline) was loaded into each well of the plates. The plates were incubated 2 h at room temperature or overnight at 4° C. Saturation: 40 μl/well PBS 0.1% Tween 2% milk were incubated for 1 hour at room temperature or overnight at 4° C. 3 washes with 50 μl of PBS+0.1% Tween were performed and 3 washes with 50 μl PBS were performed.

The phages were pre-incubated for 20 minutes at room temperature. 48 μl of phages and 17 μl PBS+0.1% Tween+2% milk were added to each well. The phages were incubated 120 minutes at room temperature. The wells were then washed 3× with 50 μl of PBS+0.1% Tween and washed 3× with 50 μl PBS.

12.5 μl/well Myc was added at a dilution of 1/500 in PBS+0.1% Tween+2% milk, and was incubated for 1 hour at room temperature. Each well was then washed with 50 μl of PBS+0.1% Tween and washed twice with 50 μl PBS.

12.5 μl/well secondary antibody anti-mouse HRP was added to each well at a dilution of 1/5000 in PBS+0.1% Tween+2% milk and incubated for 40 minutes at room temperature. The wells were washed 3× with 50 μl of PBS+0.1% Tween and 3 washes with 50 μl PBS.

Detection: TMB (transparent plates) or ECL (White plates), 25 μl/well TMB (3,3′,5,5′-Tetramethylbenzidine) was added. The reaction was stopped by adding 25 μl of sulfuric acid 2N (1 M). A spectrometer reading at DO=450 nm was then taken.

### Example 1

### Identification of Single Domain Antibodies Recognizing LF Protein

A series of assays were performed to identify anti-LF single-domain antibodies from a V_{H}H library, and to validate the anti-LF antibodies by Phage ELISA and V_{H}H ELISA assays.

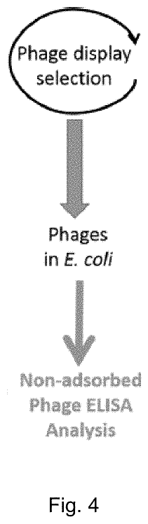

Three rounds of phage display selection were conducted using biotinylated LF protein. A synthetic hydroxysteroid dehydrogenase type 2 (HSD2) single heavy chain variable domain antibody (“V_{H}H”) library of 3×10⁹ clones (provided by Hybrigenics Services, Paris, France) was expressed at the surface of M13 phage. The phage display allowed the selection of V_{H}Hs recognizing the non-adsorbed antigen in a native form. Selected V_{H}Hs were validated in non-adsorbed phase ELISA and V_{H}H ELISA assays, as set forth in the schematic of FIG. 4.

Antigen Preparation:

A non-related protein (His-SNAP-Halo-Biotin) was used in an initial round of phage display to delete the library from unspecific binding molecules.

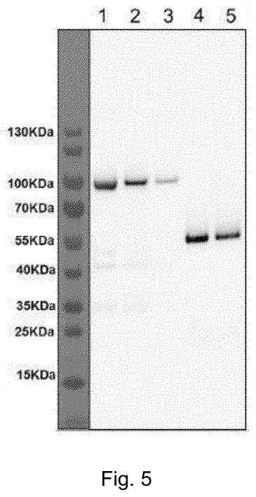

Prior to the phage display selection, LF-Biotin and His-SNAP-Halo-Biotin were bound to streptavidin magnetic beads (DYNABEADS® M-280 Streptavidin, provided by Life Technologies, Carlsbad, Calif.) with a 50 nM final concentration of biotinylated protein for the first round, and a 10 nM final concentration of biotinylated protein for the second and third rounds. Successful binding of the biotinylated proteins on the streptavidin beads was confirmed in an SDA-PAGE/Western blot using a streptavidin-HRP conjugate (provided by Thermo Fisher Scientific, Waltham, Mass.). FIG. 5 illustrates a Western blot of biotinylated proteins fixed on streptavidin magnetic beads. As shown, Lane 1=1182 ng of LF-Biotin without beads; Lane 2=1182 ng of LF-Biotin on beads; Lane 3=258 ng of LF-Biotin on beads; Lane 4=155 ng of His-SNAP-Halo-Biotin without beads; Lane 5=155 ng of His-SNAP-Halo-Biotin on beads. The Western blot indicated that the biotinylated proteins were successfully bound to the magnetic beads.

Phage Display Selection (3 Rounds):

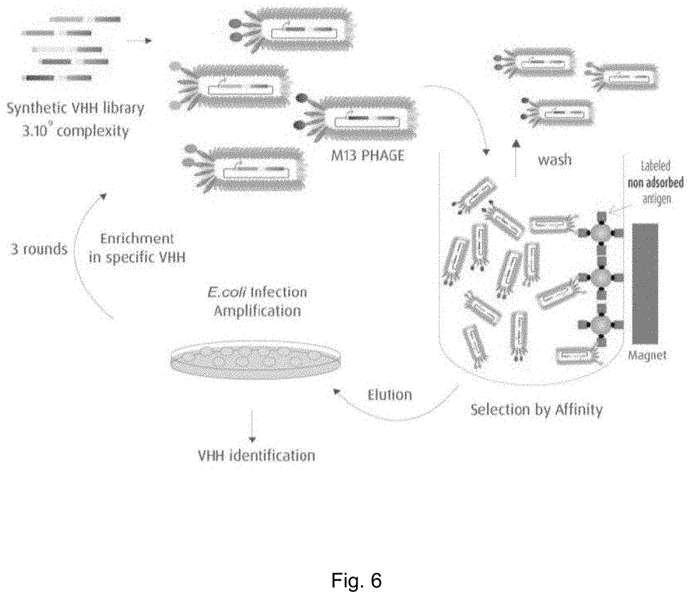

The HS2D Ab Phage Display library presenting 3×10⁹ V_{H}Hs was first incubated with the His-SNAP-Halo-Biotin beads to remove unspecific binders. The unbound V_{H}Hs expressed as an E. coli supernatant were then incubated with the LF-Biotin beads. A total of three rounds of phage display were performed, as shown in the schematic of FIG. 6. The depletion step was repeated before each Phage Display round to remove non-specific V_{H}Hs.

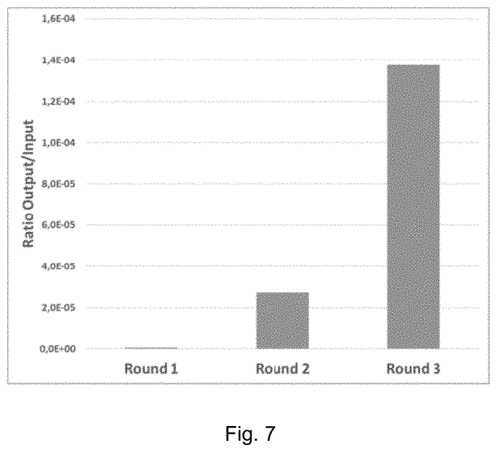

To determine the binding of specific anti-LF V_{H}Hs during each round of Phage Display, an output/input ratio was determined by measuring the concentration of specific anti-LF V_{H}Hs before and after 3 consecutive rounds of phage display selection. The results are shown below in Table 2 and are illustrated graphically in FIG. 7. It was determined that the third round exhibited the greatest binding compared to the first and second rounds.

TABLE 2
Input/Output Values and Ratios During
3 Consecutive Rounds of Phage Display

| | Round 1 | Round 2 | Round 3 |
|---|---|---|---|
| Input | 8.1E+11 | 1.3E+12 | 1.5E+12 |
| Output | 4.9E+5 | 3.7E+7 | 2.1E+8 |
| Output/Input | 6.1E−07 | 2.7E−5 | 1.4E−4 |

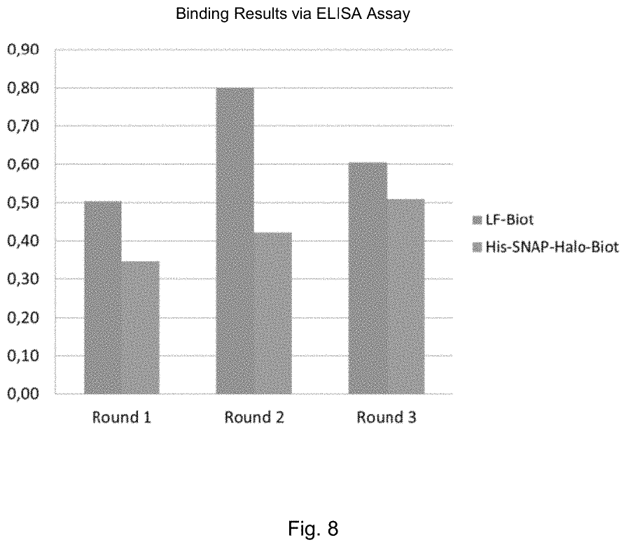

An ELISA assay on the pool of each round was performed to further determine binding during the 3 consecutive rounds of phase display selection. The results are illustrated graphically in FIG. 8. As shown, progressive binding during the cycle indicates that the Phage Display conditions are appropriate. At the end of the 3rd round of Phage Display, 183 E. coli clones were randomly selected for further analysis.

V_{H}H Validation Using Non-Adsorbed Phage ELISA

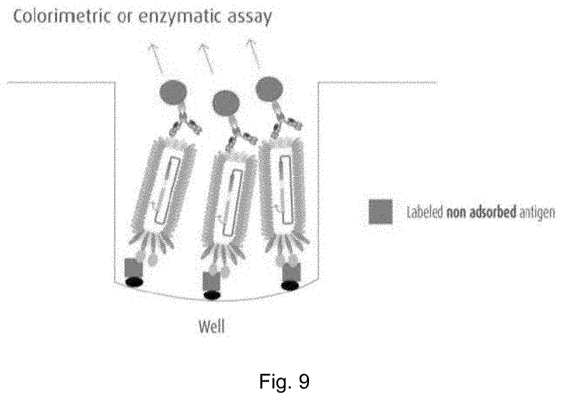

The LF-Biotin binding of the 183 V_{H}H clones selected after three rounds of Phage Display was tested using a non-adsorbed phage ELISA assay that allowed for the proper folding of the native LF protein, as illustrated in the schematic of FIG. 9.

The phages produced from each E. coli clone were used in a 384 well plate ELISA assay with HRP-conjugated anti-M13 antibody (provided by GE Healthcare, Chicago, Ill.) and a colorimetric substrate (TMB, Tetramethylbenzidine, Thermo Fisher Scientific, Waltham, Mass.). The results of the first ELISA assay with random clones are set forth in Table 3, below. V_{H}H clones that exhibited a significant ELISA signal in the presence of LF-Biotin and a very low signal in the presence of His-SNAP-Halo-Biotin were considered as specific LF binders and were selected for sequence analysis.

TABLE 3
Results of Non-Adsorbed ELISA
of 183 Randomly Selected Clones

| | No. | Total | Percentage |
|---|---|---|---|
| Positive Hit | 30 | 30 | 16.4% |
| Weak Positive Hit | 0 | | |
| Negative Hit | 153 | 153 | 83.6% |

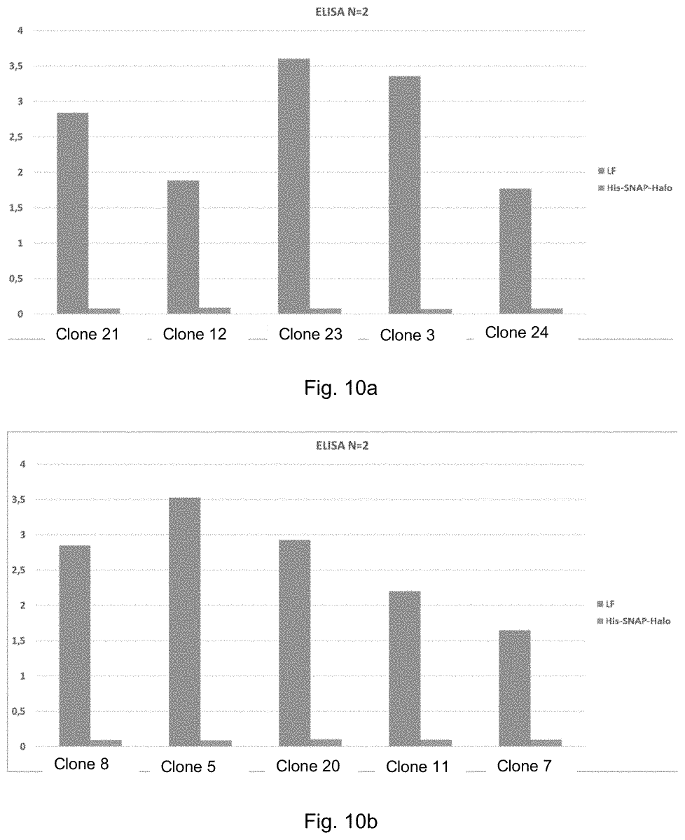

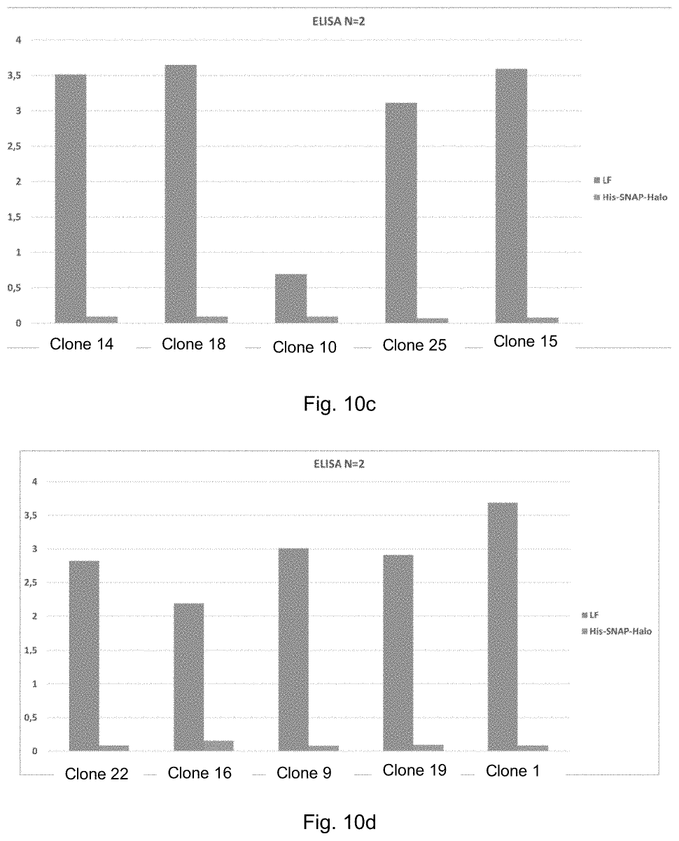

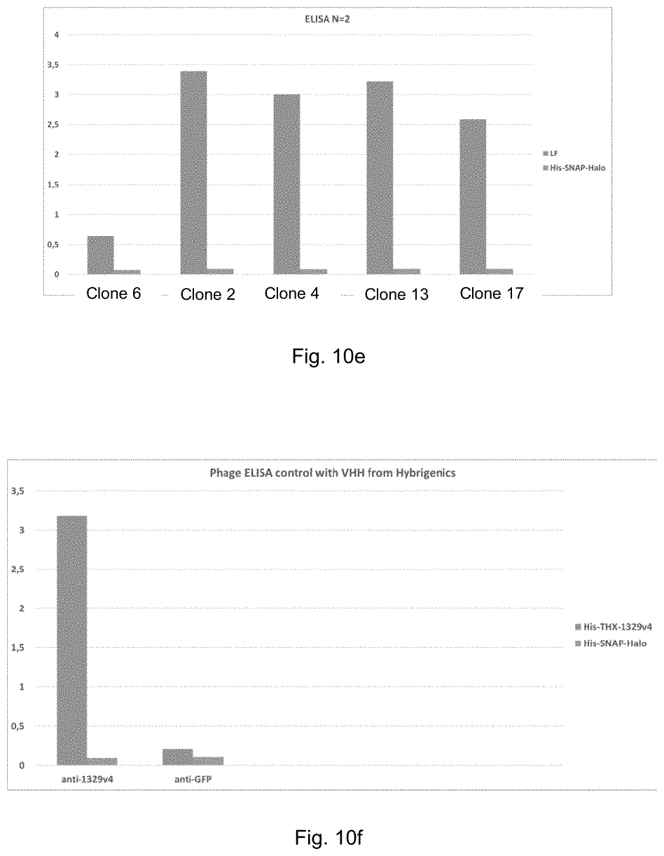

The sequencing of the positive clones revealed the presence of 25 available different positive V_{H}Hs. They were reconfirmed (N=2) by non-adsorbed ELISA, as shown in FIGS. 10a-10f.

V_{H}H Validation Using Non-Adsorbed V_{H}H ELISA

The LF-Biotin binding of the 25 selected V_{H}H clones was tested using non-adsorbed V_{H}H ELISA that allows for the proper folding of the native LF protein and to select the best candidates (FIG. 9).

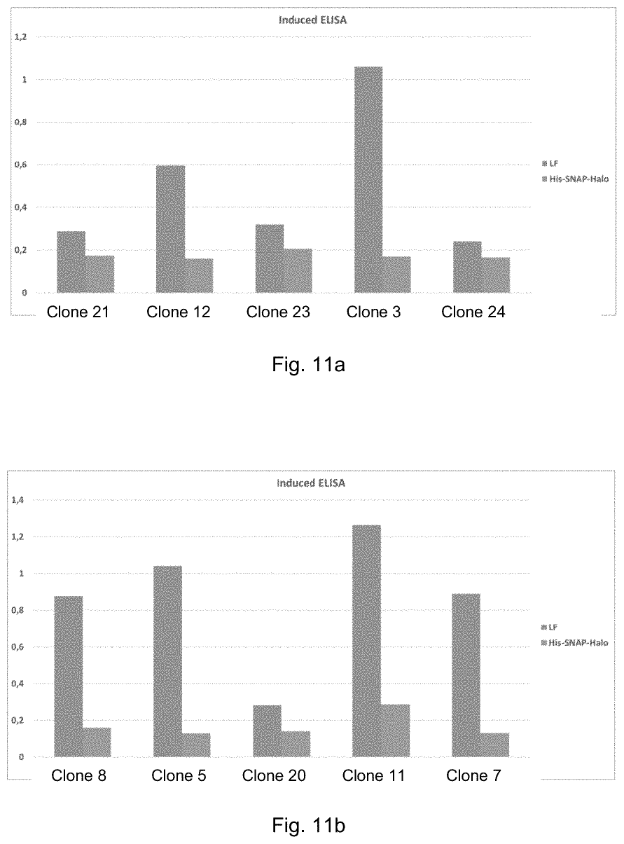

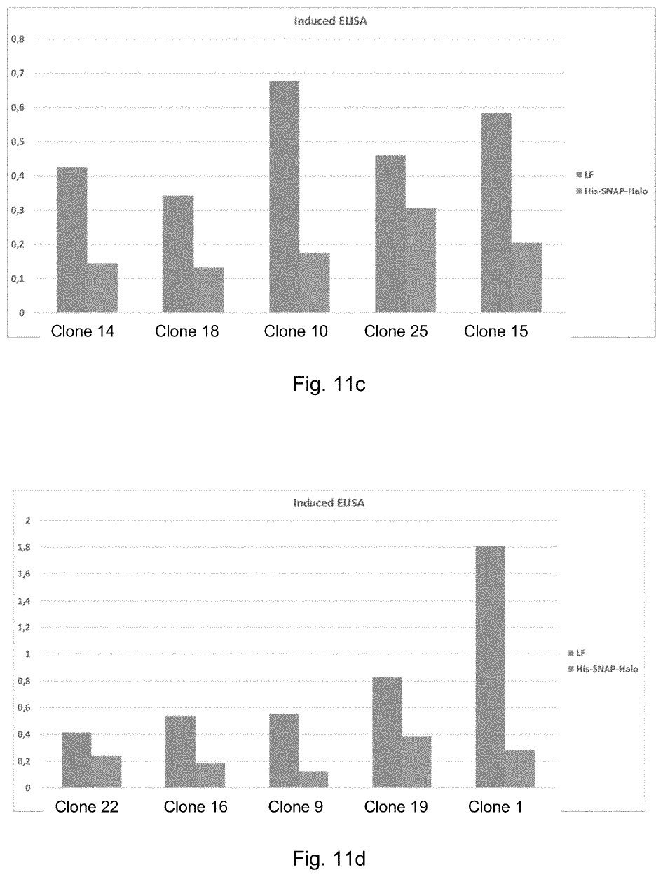

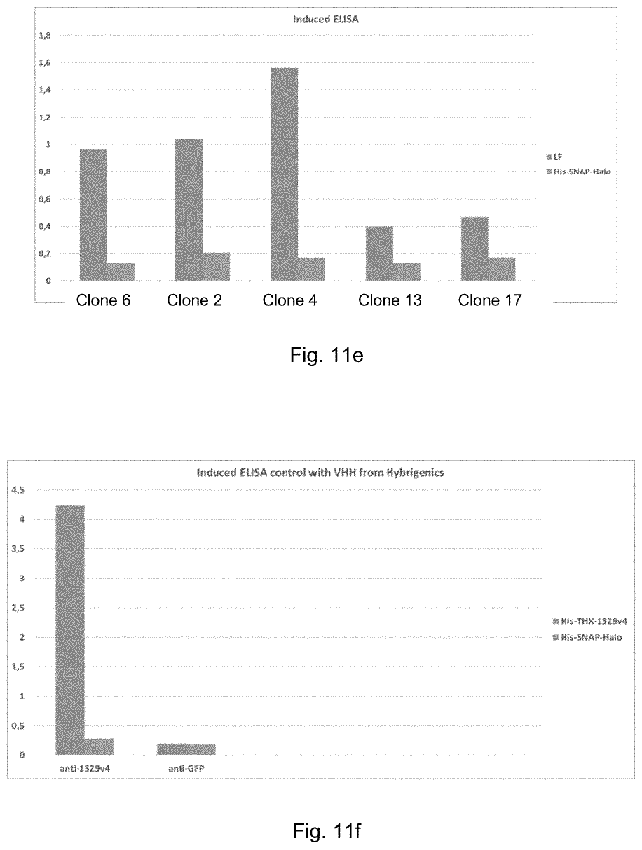

The V_{H}H-myc produced by IPTG induction from each E. coli clone were used in a 384 well plate ELISA with anti-myc (provided by Invitrogen Corp., Carlsbad, Calif.), HRP-conjugated anti-mouse (provided by Novus Biologicals, Littleton, Colo.) and a calorimetric substrate (TMB, Tetramethylbenzidine, Thermo Fisher Scientific, Waltham, Mass.). V_{H}H clones with a significant ELISA signal in the presence of LF-Biotin and a very low signal in the presence of His-SNAP-Halo-Biotin were considered as specific LF binders and were selected (FIGS. 11a-11f).

Sequences and Clones Delivery

25 clones were selected for further testing. As shown in Table 4, the phage ELISA ratio of signal LF/Signal HIS-SNAP-Halo for all 25 clones were positive. The Induced ELISA ratio of signal LF/signal His-SNAP-Halo for clones 1-13 was positive, clones 14-19 was weakly positive, and for clones 20-25 was negative.

Clone 16 had an Amber codon and can be corrected because only the Phage can read this codon like a Glutamic Acid. This codon is read like a STOP codon in the other systems (mammals or classical bacterial strain).

TABLE 4
Summary of Clone Redundancy

| | | Phage ELISA | Induced ELISA | |
|---|---|---|---|---|
| | | ratio signal | ratio Signal | |
| Clone | | LF/signal | LF/signal | Amber |
| name | Redundancy | His-SNAP-Halo | His-SNAP-Halo | Codon |
| 1 | 3 | 45.5 | 6.3 | No |
| 2 | 3 | 36.5 | 5.0 | No |
| 3 | 2 | 47.2 | 6.3 | No |
| 4 | 1 | 34.6 | 9.2 | No |
| 5 | 1 | 41.5 | 8.0 | No |
| 6 | 1 | 8.9 | 7.3 | No |
| 7 | 1 | 17.0 | 6.8 | No |
| 8 | 1 | 30.2 | 5.5 | No |
| 9 | 1 | 38.1 | 4.6 | No |
| 10 | 1 | 7.7 | 3.9 | No |
| 11 | 1 | 22.6 | 3.8 | No |
| 12 | 1 | 21.9 | 3.7 | No |
| 13 | 1 | 34.6 | 3.0 | No |
| 14 | 1 | 39.0 | 2.9 | No |
| 15 | 1 | 46.6 | 2.9 | No |
| 16 | 1 | 14.6 | 2.9 | Yes |
| 17 | 1 | 28.4 | 2.7 | No |
| 18 | 1 | 40.9 | 2.6 | No |
| 19 | 1 | 31.6 | 2.2 | No |
| 20 | 1 | 28.4 | 2.0 | No |
| 21 | 1 | 35.9 | 1.7 | No |
| 22 | 1 | 34.9 | 1.7 | No |
| 23 | 1 | 46.2 | 1.6 | No |
| 24 | 1 | 22.1 | 1.5 | No |
| 25 | 1 | 44.5 | 1.5 | No |

10 clones were collected from the screening according to redundancy, ELISA assay results, and sequencing results. The 10 clones selected were clones 1, 6, 3, 4, 5, 8, 7, 9, 2, and 10. The amino acid sequence of each clone was identified as clone 1 (SEQ ID NO: 1); clone 6 (SEQ ID NO: 2); clone 3 (SEQ ID NO: 3); clone 4 (SEQ ID NO: 4); clone 5 (SEQ ID NO: 5); clone 8 (SEQ ID NO: 6); clone 7 (SEQ ID NO: 7); clone 9 (SEQ ID NO: 8); clone 2 (SEQ ID NO: 9); clone 10 (SEQ ID NO: 10).

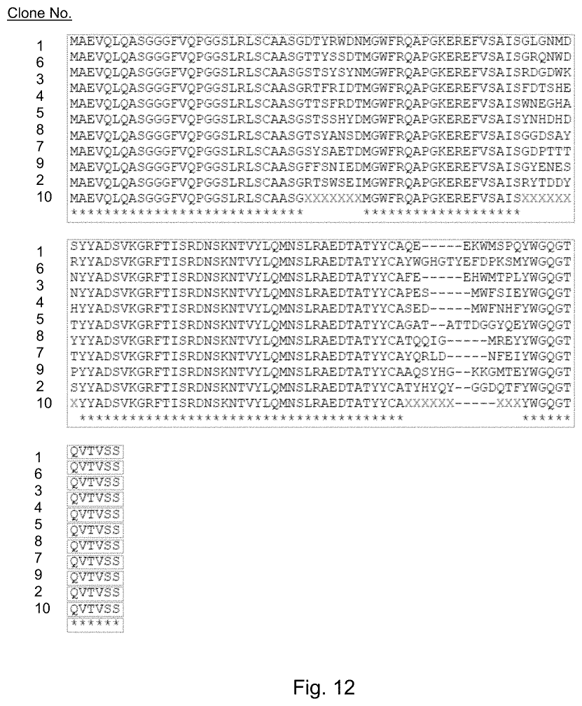

The alignment of the 10 selected clones are shown in FIG. 12. The reference is a nanobody with CFR region replaced by “X.”

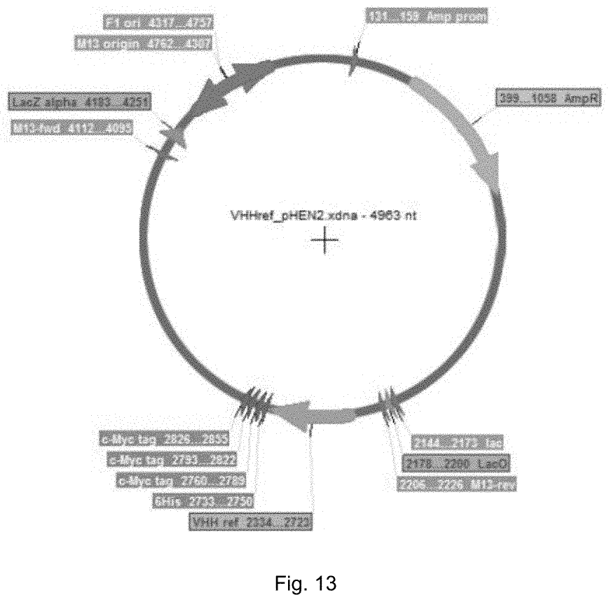

A map of V_{H}Hr of pHEN2 is shown in FIG. 13.

### Example 2

### Identification of Single-Domain Antibodies Recognizing EF Protein

A series of assays was conducted to identify anti-EF single-domain antibodies from a V_{H}H library. The anti-EF single domain antibodies were validated by Phage ELISA assay.

Three rounds of Phage Display selection were conducted using biotinylated EF protein. A synthetic HS2D Ab V_{H}H library of 3×10⁹ clones was expressed at the surface of M13 phage. The phage display allowed the selection of V_{H}Hs recognizing the non-adsorbed antigen in a native form. Selected V_{H}Hs were validated in non-adsorbed Phage ELISA.

Antigen Preparation

As described in Example 1, His-SNAP-Halo-Biotin was used in an initial round of Phage Display to deplete the library from unspecific binding molecules.

Prior to the Phage Display selection, EF-Biotin and His-SNAP-Halo-Biotin were bound to streptavidin magnetic beads (Dynabeads® M-280 Streptavidin, provided by Life Technologies, Carlsbad, Calif.) with a 50 nM final concentration of biotinylated protein for the first round and 10 nM final concentration of biotinylated protein for the second and third rounds. Successful binding of the biotinylated proteins on the streptavidin beads was confirmed in an SOS-PAGE/Western Blot using a Streptavidin-HRP conjugate (provided by Thermo Fisher Scientific, Waltham, Mass.).

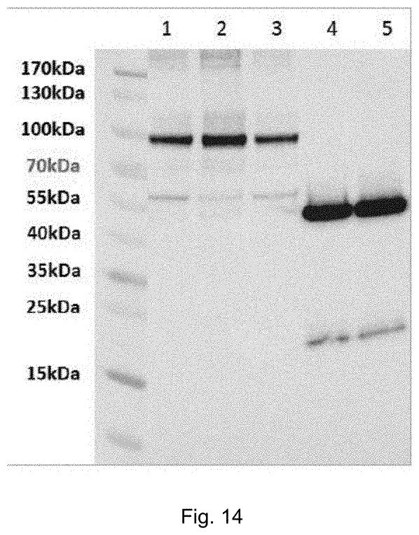

FIG. 14 illustrates a Western blot of biotinylated proteins fixed on Strepavidin magnetic beads. Lane 1: 250 ng of EF-Biotin without beads; Lane 2: 1140 ng of EF-Biotin on beads; Lane 3: 250 ng of EF-Biotin on beads; Lane 4: 155 ng of His-SNAP-Halo-Biotin without beads; Lane 5: 155 ng of His-SNAP-Halo-Biotin on beads.

Phage Display Selection (3 Rounds)

The HS2D Ab Phage Display library presenting 3×10⁹ V_{H}Hs was first incubated with the His-SNAP-Halo-Biotin beads to remove unspecific binders. The unbound V_{H}Hs expressed as an E. coli supernatant were incubated with the EF-Biotin beads. A total of three rounds of Phage Display were performed, as shown in the schematic of FIG. 6. The depletion step was repeated before each Phage Display round to remove non-specific V_{H}Hs.

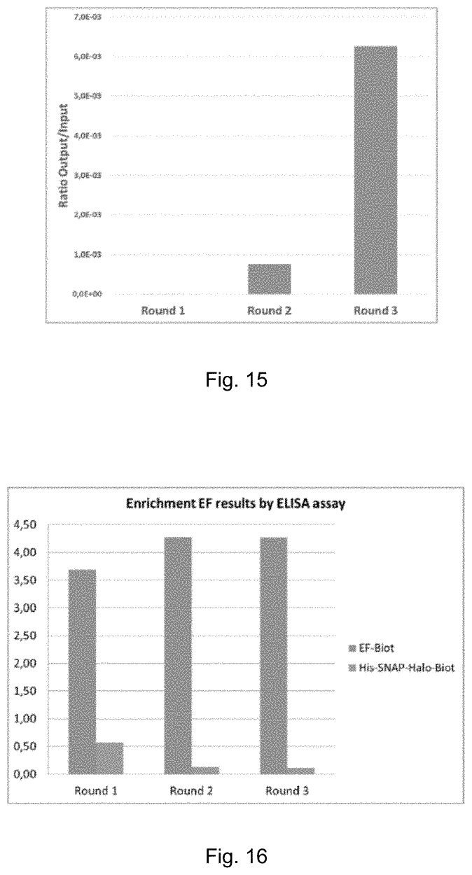

To determine binding of the specific anti-EF V_{H}Hs during each round of Phage Display, an output/input ratio was determined by measuring the concentration of specific anti-EF V_{H}Hs before and after the 3 consecutive rounds of phage display selection. The results are shown below in Table 5 and are illustrated graphically in FIG. 15.

TABLE 5
Input/Output Values and Ratios During
3 Consecutive Rounds of Phage Display

| | Round 1 | Round 2 | Round 3 |
|---|---|---|---|
| Input | 8.1E+11 | 3.5E+11 | 3.3E+11 |
| Output | 3.9E+5 | 2.6E+8 | 2.1E+9 |
| Output/Input | 4.8E−07 | 7.5E−4 | 6.3E−3 |

An ELISA assay was performed on the pool of each round to further determine binding during the 3 consecutive rounds of phase display selection. The results are indicated graphically in FIG. 16. As shown, progressive binding during the cycle shows that the Phage Display conditions are appropriate. At the end of the 3rd round of Phage Display, 90 E. coli clones were selected randomly.

### V_{H}H Validation Using Non-Adsorbed Phage ELISA

The EF-Biotin binding of the 90 V_{H}H clones selected after three rounds of Phage Display was tested using a non-adsorbed phage ELISA assay that allowed for the proper folding of the native EF protein, as illustrated in the schematic of FIG. 9.

The phages produced from each E. coli clone were used in a 384 well plate ELISA with HRP-conjugated anti-M13 antibody (provided by GE Healthcare, Chicago, Ill.) and a colorimetric substrate (TMB, Tetramethylbenzidine, Thermo Fisher Scientific, Waltham, Mass.). The results of the first ELISA assay with random clones are set forth below in Table 6. V_{H}H clones with a significant ELISA signal in the presence of EF-Biotin and a very low signal in the presence of His-SNAP-Halo-Biotin were considered as specific EF binders and were selected for sequence analysis.

TABLE 6
Results of Non-Adsorbed ELISA of 90 Randomly Selected Clones

| ID. | No. | Total | Percentage |
|---|---|---|---|
| Positive Hit | 78 | 82 | 91.1% |
| Weak Positive Hit | 4 | | |
| Negative Hit | 8 | 8 | 8.9% |

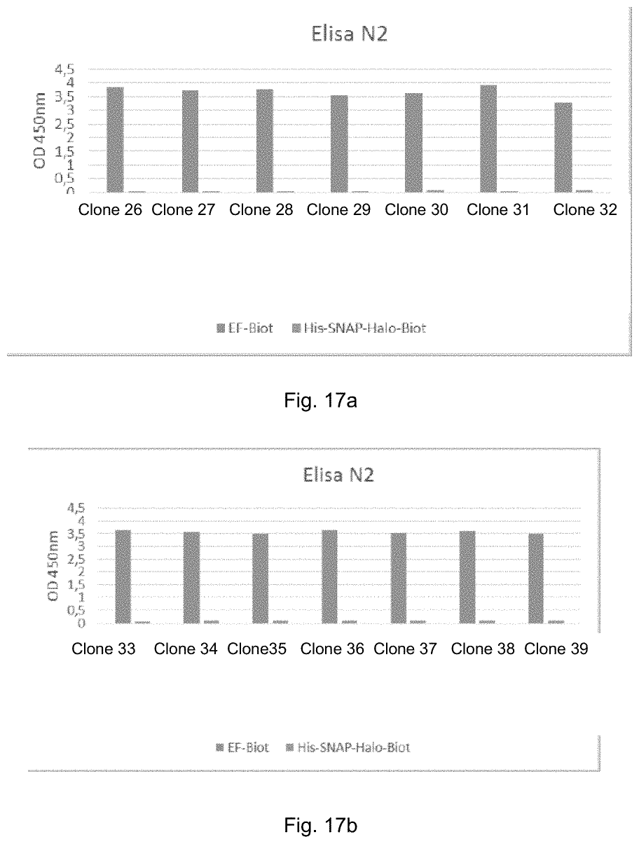

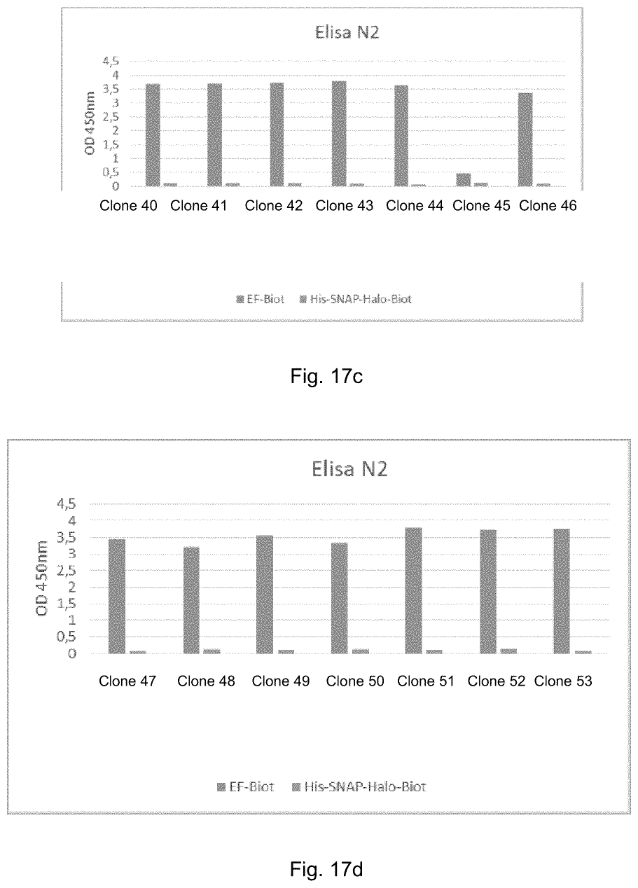

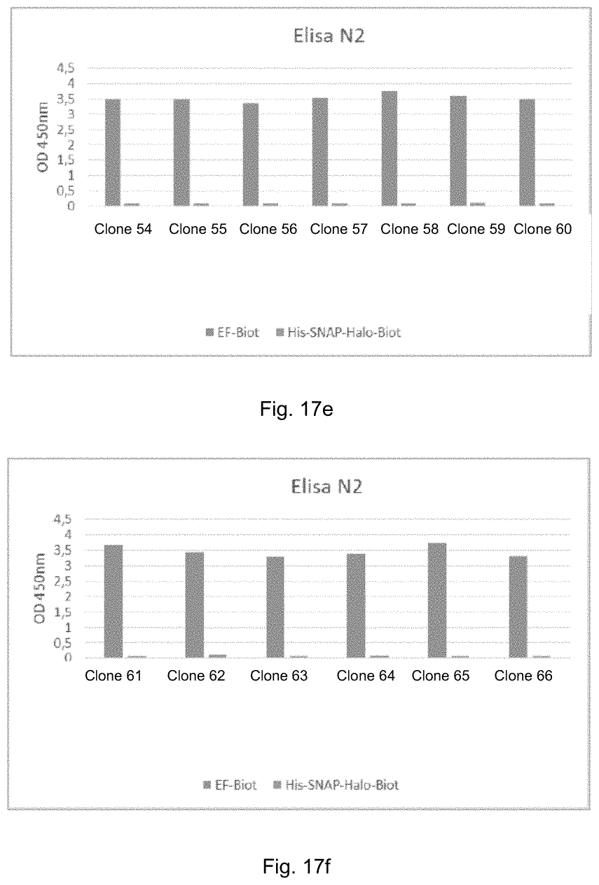

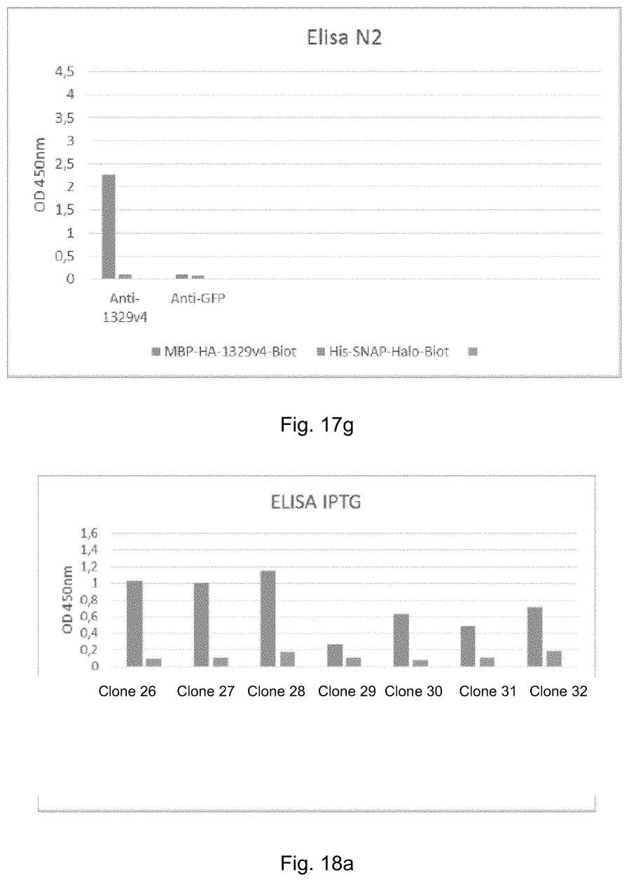

The sequencing of the positive clones revealed the presence of 41 available different positive V_{H}Hs. The selected V_{H}Hs were reconfirmed (N=2) by non-adsorbed ELISA, as shown in FIGS. 17a-17g.

V_{H}H Validation Using Non-Adsorbed V_{H}H ELISA

The EF-Biotin binding of the 41 selected V_{H}H clones was tested using a non-adsorbed V_{H}H ELISA that allowed for the proper folding of the native EF protein and to select the best candidates (FIG. 9).

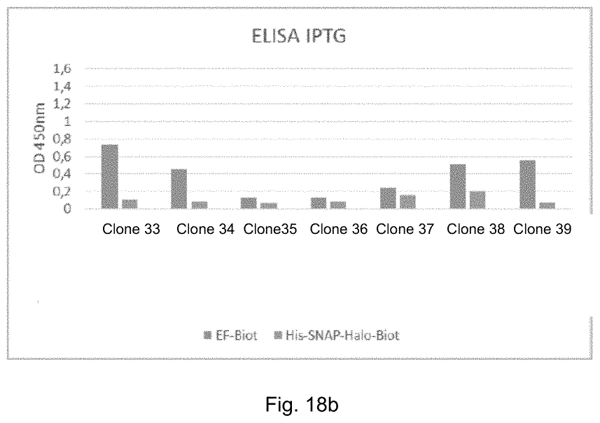

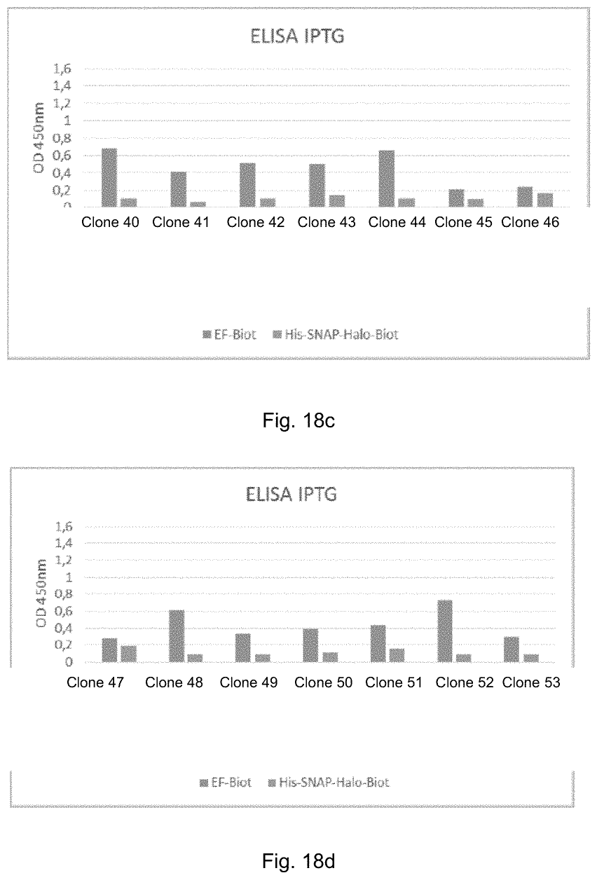

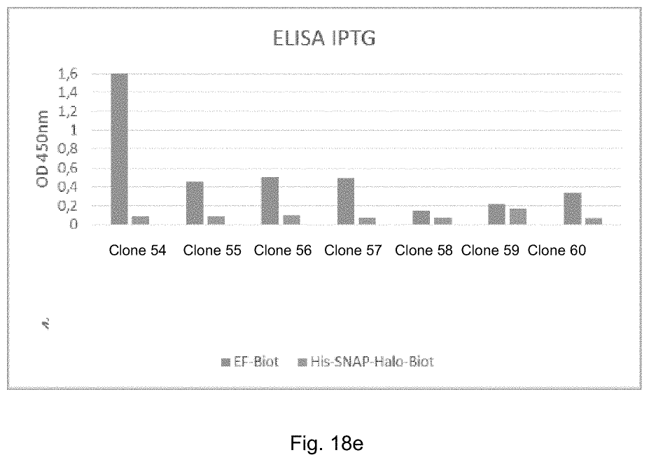

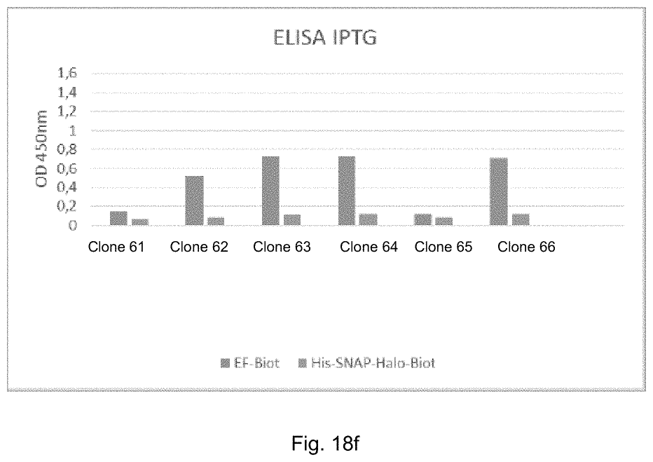

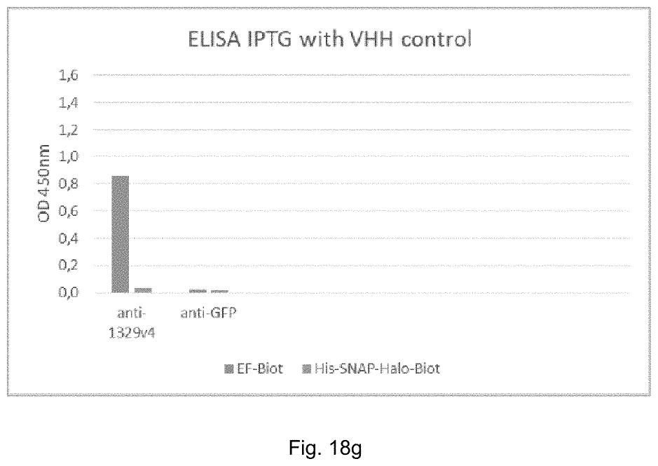

The V_{H}H-myc produced by IPTG induction from each E. coli clone were used in a 384 well plate ELISA with anti-myc (provided by Invitrogen Corp.. Carlsbad, Calif.), HRP-conjugated anti-mouse (provided by Novus Biologicals, Littleton, Colo.) and a colorimetric substrate (TMB, Tetramethylbenzidine, Thermo Fisher Scientific, Waltham, Mass.). V_{H}H clones with a significant ELISA signal in the presence of EF-Biotin and a very low signal in the presence of His-SNAP-Halo-Biotin were considered to be specific EF binders and were selected (FIGS. 18a-g).

Sequences and Clones Delivery

10 clones were selected for further testing based on redundancy, ELISA assay results, and the sequencing results (clones 26, 28, 40, 41, 42, 44, 49, 57, 60, and 66). The phage ELISA Ag+/Ag− and phage IPTG Ag+/Ag− were all positive.

TABLE 7
Summary of Clone Redundancy

| | | Phage ELISA | Phage IPTG |
|---|---|---|---|
| Clone | Redundancy | Ag+/Ag− | Ag+/Ag− |
| 26 | 2 | 51.3 | 10.2 |
| 28 | 3 | 44.5 | 6.6 |
| 40 | 2 | 31.5 | 6.2 |
| 41 | 2 | 36.7 | 5.7 |
| 42 | 15 | 36.2 | 5.0 |
| 44 | 2 | 52.7 | 6.1 |
| 49 | 2 | 36.7 | 3.6 |
| 57 | 2 | 40.1 | 6.3 |
| 60 | 2 | 43.6 | 4.9 |
| 66 | 2 | 51.8 | 5.8 |

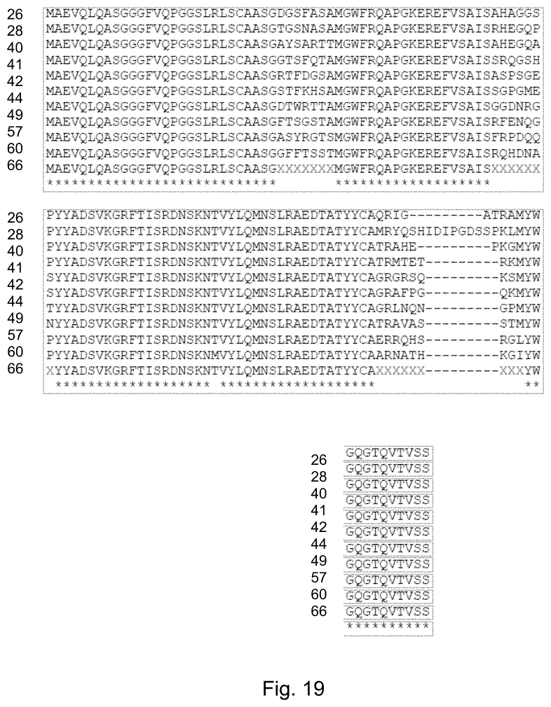

The alignment of the 10 selected clones are shown in FIG. 19. The amino acid sequence of each clone was identified as clone 26 (SEQ ID NO: 11); clone 28 (SEQ ID NO: 12); clone 40 (SEQ ID NO: 13); clone 41 (SEQ ID NO: 14); clone 42 (SEQ ID NO: 15); clone 44 (SEQ ID NO: 16); clone 49 (SEQ ID NO: 17); clone 57 (SEQ ID NO: 18); clone 60 (SEQ ID NO: 19); clone 66 (SEQ ID NO: 20).

The reference is a nanobody with CFR region replaced by “X.”

### Example 3

### Identification of Single Domain Antibodies Recognizing TSST-1 Protein

A series of assays were performed to identify anti-TSST-1 single-domain antibodies from a V_{H}H library, and to validate the anti-TSST-1 antibodies by non-adsorbed phase ELISA.

Three rounds of Phage Display selection were carried out using biotinylated TSST1. A synthetic HS2D Ab V_{H}H library of 3×10⁹ clones was expressed at the surface of M13 phage. The Phage Display allowed the selection of V_{H}Hs recognizing the non-adsorbed antigen in a native form. Selected V_{H}Hs were validated in non-adsorbed Phage ELISA.

VHH Selection in vitro and Phase ELISA Validation

After three rounds of Phage Display, 90 V_{H}Hs were selected and analyzed for binding to TSST1. However, none were positive in non-adsorbed phage ELISA.

VHH Selection in Yeast

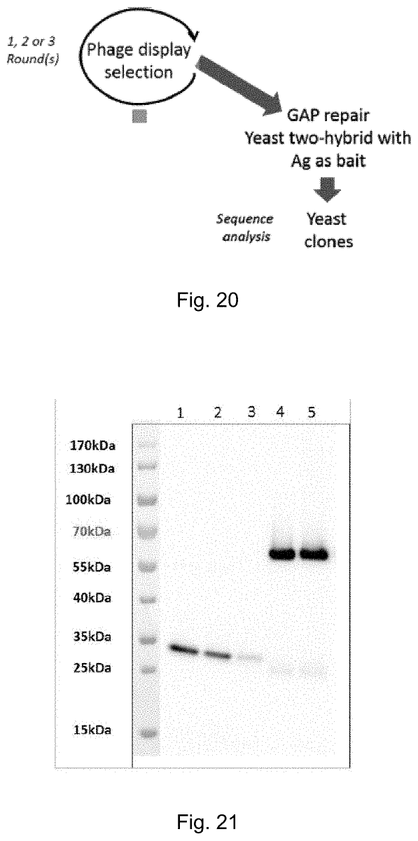

The coding sequence for S. aureus TSST-1 (aa 41-234) was FOR amplified and cloned into pB27 as a C terminal fusion to LexA (LexA-TSST1), as shown in the schematic of FIG. 20.

During the First Round of screening, 1.1×10⁶ nanobodies were collected. A yeast library from Round 1 was created with 2.5×10⁵ yeast cells complexity. During the 2YH screen against TSST1 bait, 1 million interactions were tested, and 190 positive clones were selected. The sequencing of the clones revealed the presence of about 28 different clones.

Antigen Preparation

As described in Example 1, His-SNAP-Halo-Biotin was used in an initial round of Phage Display to deplete the library from unspecific binding molecules.

Prior to the Phage Display selection, TSST1-Biotin and His-SNAP-Halo-Biotin were bound to Streptavidin Magnetic Beads (Dynabeads® M-280 Streptavidin, provided by Life Technologies, Carlsbad, Calif.) with a 50 nM final concentration of biotinylated protein for the first round and 10 nM final concentration of biotinylated protein for the second and third rounds. Successful binding of the biotinylated proteins on the streptavidin beads was confirmed in an SOS-PAGE/Western Blot using a Streptavidin-HRP conjugate (provided by Thermo Fisher Scientific, Waltham, Mass.).

FIG. 21 illustrates a Western blot of biotinylated proteins fixed on Streptavidin magnetic beads. Lane 1: 302 ng of TSST1-Biotin without beads (50 nM); Lane 2: 302 ng of TSST1-Biotin on the beads (50 nM for the Round 1); Lane 3: 66 ng of TSST1-Biotin on beads (10 nM for the rounds 2 and 3); Lane 4: 155 ng of His-SNAP-Halo-Biotin without beads (10 nM for all the rounds); Lane 5: 155 ng of His-SNAP-Halo-Biotin on beads (10 nM for the all the rounds).

Phage Display Selection

The HS2D Ab Phage Display library presenting 3×10⁹ V_{H}Hs was first incubated with the His-SNAP-Halo-Biotin beads to remove unspecific binders. The unbound V_{H}Hs expressed as an E. coli supernatant were incubated with the TSST-1-Biotin beads. A total of three rounds of Phage Display were performed, as shown in the schematic of FIG. 6. The depletion step was repeated before each Phage Display round to remove unspecific V_{H}Hs.

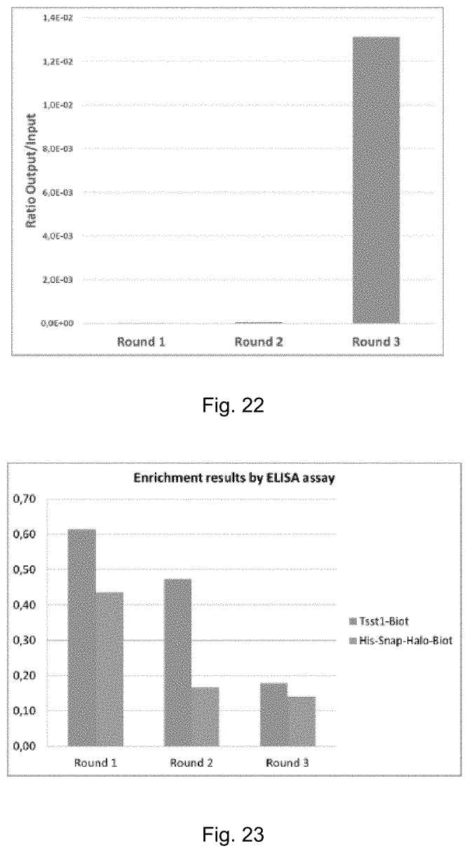

To determine the binding of specific anti-TSST-1 V_{H}Hs during each round of Phage Display, an output/input was determined by measuring the concentration of specific anti-TSST-1 V_{H}Hs before and after the 3 consecutive rounds of phage display selection. The results are shown below in Table 8 and are illustrated graphically in FIG. 22.

TABLE 8
Input/Output Values and Ratios During
3 Consecutive Rounds of Phage Display

| | Round 1 | Round 2 | Round 3 |
|---|---|---|---|
| Input | 8.1E+11 | 5.9E+12 | 1.6E+11 |
| Output | 2.1E+5 | 4.3E+7 | 2.1E+9 |
| Output/Input | 2.6E−07 | 7.3E−5 | 1.3E−2 |

An ELISA assay on the pool of each round was performed, as shown in FIG. 23. Progressive enrichment during the cycle shows that the Phage Display conditions are appropriate (Table 8). At the end of the 3rd round of Phage Display, 90 E. coli clones were randomly selected and analyzed by non-adsorbed phage ELISA.

V_{H}H Validation using Non-Adsorbed Phage ELISA

The binding of the 90 V_{H}H selected clones to TSST-1 was tested using anon-adsorbed phage ELISA assay that allows for the proper folding of the native TSST-1 protein, as described in Example 1.

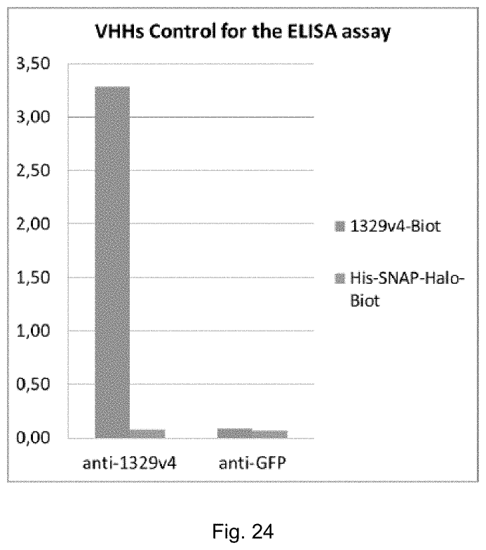

The phages produced from each E. coli clone were used in a 384 well plate ELISA with HRP-conjugated anti-M13 antibody (provided by GE Healthcare, Chicago, Ill.) and a colorimetric substrate (TMB, TetraMethylBenzidine, Thermo Fisher Scientific, Waltham, Mass.). The first ELISA with random clones are presented in Table 9. V_{H}H clones with a significant ELISA signal in the presence of TSST-1-Biotin and a very low signal in the presence of His-SNAP-Halo-Biotin were considered as specific TSST-1 binders. As indicated in Table 9, none of the clones were positive for TSST-1 protein. The data is shown graphically in FIG. 24.

TABLE 9
Results of Non-Adsorbed ELISA of 90 Randomly Selected Clones

| ID. | No. | Total | Percentage |
|---|---|---|---|
| Positive Hit | 0 | 0 | 0% |
| Weak Positive Hit | 0 | | |
| Negative Hit | 90 | 90 | 100% |

V_{H}H Selection

The coding sequence for S. aureus TSST11 (aa 41-234) was PCR amplified and cloned into pB27 as a C-terminal fusion to LexA (LexA-TSST-1).

The construct was checked by sequencing the entire insert and used as a bait to screen the Yeast Library from round 1 of phage display. pB27 derives from the original pBTM116 (Vojtek and Hollenberg, 1995) plasmid.

During the first round of screening, 1.1×10⁶ nanobodies were collected. A Yeast Library from this round was created with 2.5×10⁶ yeast cells complexity.

A test screen with TSST1 using the LexA system was completed. The protein was determined not to be toxic, but it was little auto activating. As a result, a selection medium with 20 mM of 3-Aminotriazol for the final screen. 1 million interactions were tested. In total, 190 positive clones were selected and sequenced, revealing the presence of about 28 different clones (clones 67-94). A summary of the clone redundancy is given in Table 10.

TABLE 10
Summary of Clone 67- Redundancy

| | | Glycosylation |
|---|---|---|
| Clone ID | Redundancy | Site |
| 67 | 28 | No |
| 68 | 20 | No |
| 69 | 12 | No |
| 70 | 11 | No |
| 71 | 10 | No |
| 72 | 8 | No |
| 73 | 7 | No |
| 74 | 6 | No |
| 75 | 5 | No |
| 76 | 5 | No |
| 77 | 4 | No |
| 78 | 3 | No |
| 79 | 3 | No |
| 80 | 2 | No |
| 81 | 1 | No |
| 82 | 1 | No |
| 83 | 1 | No |
| 84 | 1 | No |
| 85 | 1 | No |
| 86 | 1 | No |
| 87 | 1 | No |
| 88 | 1 | No |
| 89 | 1 | No |
| 90 | 1 | No |
| 91 | 22 | Yes |
| 92 | 7 | Yes |
| 93 | 2 | Yes |
| 94 | 1 | Yes |

Sequences and Clone Delivery

10 of the 28 clones were selected based on redundancy, glycosylation site, and sequencing results (Clones 67, 76, 69, 70, 75, 68, 73, 74, 71, and 72). The amino acid sequence of each clone was identified as clone 67 (SEQ ID NO: 21); clone 76 (SEQ ID NO: 22); clone 69 (SEQ ID NO: 23); clone 70 (SEQ ID NO: 24); clone 75 (SEQ ID NO: 25); clone 68 (SEQ ID NO: 26); clone 73 (SEQ ID NO: 27); clone 74 (SEQ ID NO: 28); clone 71 (SEQ ID NO: 29); clone 72 (SEQ ID NO: 30).

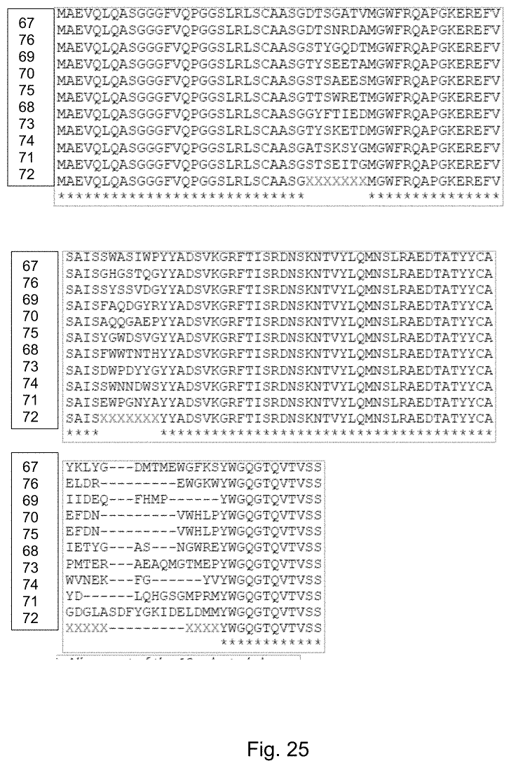

The 10 selected clones were aligned with a reference nanobody with CDR region replaced by X, as shown in FIG. 25.

What is claimed is:

1. A nanobody comprising a V_{H}H domain of a camelid heavy-chain antibody or a VNAR domain of a cartilaginous fish heavy chain antibody, wherein the nanobody is capable of neutralizing one or more bacterial toxins.

2. The nanobody of claim 1, wherein the nanobody is an anti-LF nanobody comprising at least 85% sequence identity to the amino acid sequence set forth in one or more of SEQ ID NO: 1-10.

3. The nanobody of claim 1, wherein the nanobody is an anti-EF nanobody comprising at least 85% sequence identity to the amino acid sequence set forth in one or more of SEQ ID NO: 11-20.

4. The nanobody of claim 1, wherein the nanobody is an anti-TSST-1 nanobody comprising at least 85% sequence identity to the amino acid sequence set forth in one or more of SEQ ID NO: 21-30.

5. The nanobody of claim 1, wherein the bacterial toxins are associated with anthrax exposure or toxic shock syndrome.

6. The nanobody of claim 1, wherein the nanobody is humanized.

7. A pharmaceutical composition comprising or consisting essentially of a nanobody comprising a V_{H}H domain of a camelid heavy-chain antibody or a VNAR domain of a cartilaginous fish heavy chain antibody, wherein the nanobody is capable of neutralizing one or more bacterial toxins.

8. The pharmaceutical composition of claim 7, wherein the nanobody is an anti-LF nanobody comprising at least 85% sequence identity to the amino acid sequence set forth in one or more of SEQ ID NO: 1-10.

9. The pharmaceutical composition of claim 7, wherein the nanobody is an anti-EF nanobody comprising at least 85% sequence identity to the amino acid sequence set forth in one or more of SEQ ID NO: 11-20.

10. The pharmaceutical composition of claim 7, wherein the nanobody is an anti-TSST-1 nanobody comprising at least 85% sequence identity to the amino acid sequence set forth in one or more of SEQ ID NO: 21-30

11. The pharmaceutical composition of claim 7, further comprising a pharmaceutically acceptable carrier.

12. The pharmaceutical composition of claim 7, wherein the bacterial toxins are associated with anthrax exposure or toxic shock syndrome.

13. The pharmaceutical composition of claim 7, wherein the nanobody is humanized.

14. A method of treating, preventing, or alleviating a disorder mediated by one or more bacterial toxins, the method comprising administering an effective amount of a nanobody comprising a V_{H}H domain of a camelid heavy-chain antibody or a VNAR domain of a cartilaginous fish heavy chain antibody in an amount sufficient to neutralize the bacterial toxins.

15. The method of claim 14, wherein the nanobody is an anti-LF nanobody comprising at least 85% sequence identity to the amino acid sequence set forth in one or more of SEQ ID NO: 1-10.

16. The method of claim 14, wherein the nanobody is an anti-EF nanobody comprising at least 85% sequence identity to the amino acid sequence set forth in one or more of SEQ ID NO: 11-20.

17. The method of claim 14, wherein the nanobody is an anti-TSST-1 nanobody comprising at least 85% sequence identity to the amino acid sequence set forth in one or more of SEQ ID NO: 21-30.

18. The method of claim 14, wherein the bacterial toxins are associated with anthrax exposure or toxic shock syndrome.

19. A polypeptide comprising or consisting of one or more of SEQ ID NO: 1-30.


SEQUENCE LISTING (60 sequences)

SEQ ID NO: 1 - protein, 121 aa, Artificial Sequence
  Derived from a cartilaginous fish heavy chain antibody or a camelid heavy chain nanobody
MAEVQLQASGGGFVQPGGSLRLSCAASGDTYRWDNMGWFRQAPGKEREFVSAISGLGNMDSYYADSVKGRFTISRDNSKNTVYLQMNSLRAEDTATYYCAQEEKWMSPQYWGQGTQVTVSS

SEQ ID NO: 2 - protein, 126 aa, Artificial Sequence
  Derived from a cartilaginous fish heavy chain antibody or a camelid heavy chain nanobody
MAEVQLQASGGGFVQPGGSLRLSCAASGTTYSSDTMGWFRQAPGKEREFVSAISGRQNWDRYYADSVKGRFTISRDNSKNTVYLQMNSLRAEDTATYYCAYWGHGTYEFDPKSMYWGQGTQVTVSS

SEQ ID NO: 3 - protein, 121 aa, Artificial Sequence
  Derived from a cartilaginous fish heavy chain antibody or a camelid heavy chain nanobody
MAEVQLQASGGGFVQPGGSLRLSCAASGSTSYSYNMGWFRQAPGKEREFVSAISRDGDWKNYYADSVKGRFTISRDNSKNTVYLQMNSLRAEDTATYYCAFEEHWMTPLYWGQGTQVTVSS

SEQ ID NO: 4 - protein, 121 aa, Artificial Sequence
  Derived from a cartilaginous fish heavy chain antibody or a camelid heavy chain nanobody
MAEVQLQASGGGFVQPGGSLRLSCAASGRTFRIDTMGWFRQAPGKEREFVSAISFDTSHENYYADSVKGRFTISRDNSKNTVYLQMNSLRAEDTATYYCAPESMWFSIEYWGQGTQVTVSS

SEQ ID NO: 5 - protein, 121 aa, Artificial Sequence
  Derived from a cartilaginous fish heavy chain antibody or a camelid heavy chain nanobody
MAEVQLQASGGGFVQPGGSLRLSCAASGTTSFRDTMGWFRQAPGKEREFVSAISWNEGHAHYYADSVKGRFTISRDNSKNTVYLQMNSLRAEDTATYYCASEDMWFNHFYWGQGTQVTVSS

SEQ ID NO: 6 - protein, 124 aa, Artificial Sequence
  Derived from a cartilaginous fish heavy chain antibody or a camelid heavy chain nanobody
MAEVQLQASGGGFVQPGGSLRLSCAASGSTSSHYDMGWFRQAPGKEREFVSAISYNHDHDTYYADSVKGRFTISRDNSKNTVYLQMNSLRAEDTATYYCAGATATTDGGYQEYWGQGTQVTVSS

SEQ ID NO: 7 - protein, 121 aa, Artificial Sequence
  Derived from a cartilaginous fish heavy chain antibody or a camelid heavy chain nanobody
MAEVQLQASGGGFVQPGGSLRLSCAASGTSYANSDMGWFRQAPGKEREFVSAISGGDSAYYYYADSVKGRFTISRDNSKNTVYLQMNSLRAEDTATYYCATQQIGMREYYWGQGTQVTVSS

SEQ ID NO: 8 - protein, 121 aa, Artificial Sequence
  Derived from a cartilaginous fish heavy chain antibody or a camelid heavy chain nanobody
MAEVQLQASGGGFVQPGGSLRLSCAASGSYSAETDMGWFRQAPGKEREFVSAISGDPTTTTYYADSVKGRFTISRDNSKNTVYLQMNSLRAEDTATYYCAYQRLDNFEIYWGQGTQVTVSS

SEQ ID NO: 9 - protein, 124 aa, Artificial Sequence
  Derived from a cartilaginous fish heavy chain antibody or a camelid heavy chain nanobody
MAEVQLQASGGGFVQPGGSLRLSCAASGFFSNIEDMGWFRQAPGKEREFVSAISGYENESPYYADSVKGRFTISRDNSKNTVYLQMNSLRAEDTATYYCAAQSYHGKKGMTEYWGQGTQVTVSS

SEQ ID NO: 10 - protein, 124 aa, Artificial Sequence
  Derived from a cartilaginous fish heavy chain antibody or a camelid heavy chain nanobody
MAEVQLQASGGGFVQPGGSLRLSCAASGRTSWSEIMGWFRQAPGKEREFVSAISRYTDDYSYYADSVKGRFTISRDNSKNTVYLQMNSLRAEDTATYYCATYHYQYGGDQTFYWGQGTQVTVSS

SEQ ID NO: 11 - protein, 121 aa, Artificial Sequence
  Derived from a cartilaginous fish heavy chain antibody or a camelid heavy chain nanobody
MAEVQLQASGGGFVQPGGSLRLSCAASGAYSARTTMGWFRQAPGKEREFVSAISAHEGQAPYYADSVKGRFTISRDNSKNTVYLQMNSLRAEDTATYYCATRAHEPKGMYWGQGTQVTVSS

SEQ ID NO: 12 - protein, 121 aa, Artificial Sequence
  Derived from a cartilaginous fish heavy chain antibody or a camelid heavy chain nanobody
MAEVQLQASGGGFVQPGGSLRLSCAASGASYRGTSMGWFRQAPGKEREFVSAISFRPDQQPYYADSVKGRFTISRDNSKNTVYLQMNSLRAEDTATYYCAERRQHSRGLYWGQGTQVTVSS

SEQ ID NO: 13 - protein, 121 aa, Artificial Sequence
  Derived from a cartilaginous fish heavy chain antibody or a camelid heavy chain nanobody
MAEVQLQASGGGFVQPGGSLRLSCAASGRTFDGSAMGWFRQAPGKEREFVSAISASPSGESYYADSVKGRFTISRDNSKNTVYLQMNSLRAEDTATYYCAGRGRSQKSMYWGQGTQVTVSS

SEQ ID NO: 14 - protein, 121 aa, Artificial Sequence
  Derived from a cartilaginous fish heavy chain antibody or a camelid heavy chain nanobody
MAEVQLQASGGGFVQPGGSLRLSCAASGDTWRTTAMGWFRQAPGKEREFVSAISGGDNRGTYYADSVKGRFTISRDNSKNTVYLQMNSLRAEDTATYYCAGRLNQNGPMYWGQGTQVTVSS

SEQ ID NO: 15 - protein, 121 aa, Artificial Sequence
  Derived from a cartilaginous fish heavy chain antibody or a camelid heavy chain nanobody
MAEVQLQASGGGFVQPGGSLRLSCAASGDGSFASAMGWFRQAPGKEREFVSAISAHAGGSPYYADSVKGRFTISRDNSKNTVYLQMNSLRAEDTATYYCAQRIGATRAMYWGQGTQVTVSS

SEQ ID NO: 16 - protein, 121 aa, Artificial Sequence
  Derived from a cartilaginous fish heavy chain antibody or a camelid heavy chain nanobody
MAEVQLQASGGGFVQPGGSLRLSCAASGFTSGSTAMGWFRQAPGKEREFVSAISRFENQGNYYADSVKGRFTISRDNSKNTVYLQMNSLRAEDTATYYCATRAVASSTMYWGQGTQVTVSS

SEQ ID NO: 17 - protein, 121 aa, Artificial Sequence
  Derived from a cartilaginous fish heavy chain antibody or a camelid heavy chain nanobody
MAEVQLQASGGGFVQPGGSLRLSCAASGGFFTSSTMGWFRQAPGKEREFVSAISRQHDNAPYYADSVKGRFTISRDNSKNMVYLQMNSLRAEDTATYYCAARNATHKGIYWGQGTQVTVSS

SEQ ID NO: 18 - protein, 121 aa, Artificial Sequence
  Derived from a cartilaginous fish heavy chain antibody or a camelid heavy chain nanobody
MAEVQLQASGGGFVQPGGSLRLSCAASGGTSFQTAMGWFRQAPGKEREFVSAISSRQGSHPYYADSVKGRFTISRDNSKNTVYLQMNSLRAEDTATYYCATRMTETRKMYWGQGTQVTVSS

SEQ ID NO: 19 - protein, 121 aa, Artificial Sequence
  Derived from a cartilaginous fish heavy chain antibody or a camelid heavy chain nanobody
MAEVQLQASGGGFVQPGGSLRLSCAASGSTFKHSAMGWFRQAPGKEREFVSAISSGPGMESYYADSVKGRFTISRDNSKNTVYLQMNSLRAEDTATYYCAGRAFPGQKMYWGQGTQVTVSS

SEQ ID NO: 20 - protein, 130 aa, Artificial Sequence
  Derived from a cartilaginous fish heavy chain antibody or a camelid heavy chain nanobody
MAEVQLQASGGGFVQPGGSLRLSCAASGTGSNASAMGWFRQAPGKEREFVSAISRHEGQPPYYADSVKGRFTISRDNSKNTVYLQMNSLRAEDTATYYCAMRYQSHIDIPGDSSPKLMYWGQGTQVTVSS

SEQ ID NO: 21 - protein, 127 aa, Artificial Sequence
  Derived from a cartilaginous fish heavy chain antibody or a camelid heavy chain nanobody
MAEVQLQASGGGFVQPGGSLRLSCAASGDTSGATVMGWFRQAPGKEREFVSAISSWASIWPYYADSVKGRFTISRDNSKNTVYLQMNSLRAEDTATYYCAYKLYGDMTMEWGFKSYWGQGTQVTVSS

SEQ ID NO: 22 - protein, 121 aa, Artificial Sequence
  Derived from a cartilaginous fish heavy chain antibody or a camelid heavy chain nanobody
MAEVQLQASGGGFVQPGGSLRLSCAASGDTSNRDAMGWFRQAPGKEREFVSAISGHGSTQGYYADSVKGRFTISRDNSKNTVYLQMNSLRAEDTATYYCAELDREWGKWYWGQGTQVTVSS

SEQ ID NO: 23 - protein, 121 aa, Artificial Sequence
  Derived from a cartilaginous fish heavy chain antibody or a camelid heavy chain nanobody
MAEVQLQASGGGFVQPGGSLRLSCAASGSTYGQDTMGWFRQAPGKEREFVSAISSYSSVDGYYADSVKGRFTISRDNSKNTVYLQMNSLRAEDTATYYCAIIDEQFHMPYWGQGTQVTVSS

SEQ ID NO: 24 - protein, 121 aa, Artificial Sequence
  Derived from a cartilaginous fish heavy chain antibody or a camelid heavy chain nanobody
MAEVQLQASGGGFVQPGGSLRLSCAASGTYSEETAMGWFRQAPGKEREFVSAISFAQDGYRYYADSVKGRFTISRDNSKNTVYLQMNSLRAEDTATYYCAEFDNVWHLPYWGQGTQVTVSS

SEQ ID NO: 25 - protein, 121 aa, Artificial Sequence
  Derived from a cartilaginous fish heavy chain antibody or a camelid heavy chain nanobody
MAEVQLQASGGGFVQPGGSLRLSCAASGSTSAEESMGWFRQAPGKEREFVSAISAQQGAEPYYADSVKGRFTISRDNSKNTVYLQMNSLRAEDTATYYCAEFDNVWHLPYWGQGTQVTVSS

SEQ ID NO: 26 - protein, 124 aa, Artificial Sequence
  Derived from a cartilaginous fish heavy chain antibody or a camelid heavy chain nanobody
MAEVQLQASGGGFVQPGGSLRLSCAASGTTSWRETMGWFRQAPGKEREFVSAISYGWDSVGYYADSVKGRFTISRDNSKNTVYLQMNSLRAEDTATYYCAIETYGASNGWREYWGQGTQVTVSS

SEQ ID NO: 27 - protein, 127 aa, Artificial Sequence
  Derived from a cartilaginous fish heavy chain antibody or a camelid heavy chain nanobody
MAEVQLQASGGGFVQPGGSLRLSCAASGGYFTIEDMGWFRQAPGKEREFVSAISFWWTNTHYYADSVKGRFTISRDNSKNTVYLQMNSLRAEDTATYYCAPMTERAEAQMGTMEPYWGQGTQVTVSS

SEQ ID NO: 28 - protein, 121 aa, Artificial Sequence
  Derived from a cartilaginous fish heavy chain antibody or a camelid heavy chain nanobody
MAEVQLQASGGGFVQPGGSLRLSCAASGTYSKETDMGWFRQAPGKEREFVSAISDWPDYYGYYADSVKGRFTISRDNSKNTVYLQMNSLRAEDTATYYCAWVNEKFGYVYWGQGTQVTVSS

SEQ ID NO: 29 - protein, 124 aa, Artificial Sequence
  Derived from a cartilaginous fish heavy chain antibody or a camelid heavy chain nanobody
MAEVQLQASGGGFVQPGGSLRLSCAASGATSKSYGMGWFRQAPGKEREFVSAISSWNNDWSYYADSVKGRFTISRDNSKNTVYLQMNSLRAEDTATYYCAYDLQHGSGMPRMYWGQGTQVTVSS

SEQ ID NO: 30 - protein, 130 aa, Artificial Sequence
  Derived from a cartilaginous fish heavy chain antibody or a camelid heavy chain nanobody
MAEVQLQASGGGFVQPGGSLRLSCAASGSTSEITGMGWFRQAPGKEREFVSAISEWPGNYAYYADSVKGRFTISRDNSKNTVYLQMNSLRAEDTATYYCAGDGLASDFYGKIDELDMMYWGQGTQVTVSS

SEQ ID NO: 31 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagatacttatcgttgggataacatgggctggtttcgtcaggcccctggcaaggagagagagttcgtttccgccatctcgggtctgggtaacatggactcgtactacgctgacagcgtaaagggaagatttacaattagccgggataactccaaaaacacggtctatctccagatgaacagcctcagggccgaggacacagctacgtattactgtgcacaggaggagaagtggatgtcgcctcagtactggggacaggggacgcaggtaactgtgagtagc

SEQ ID NO: 32 - DNA, 378 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaacaacatatagttcggacacaatgggctggtttcgtcaggcccctggcaaggagagagagttcgtttccgccatctcgggtcgtcaaaactgggaccgttactacgctgacagcgtaaagggaagatttacaattagccgggataactccaaaaacacggtctatctccagatgaacagcctcagggccgaggacacagctacgtattactgtgcatattggggtcacggtacatatgagtttgaccctaagtcgatgtattggggacaggggacgcaggtaactgtgagtagc

SEQ ID NO: 33 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggatcaacttcatattcgtacaacatgggctggtttcgtcaggcccctggcaaggagagagagttcgtttccgccatctcgcgtgacggtgactggaagaactactacgctgacagcgtaaagggaagatttacaattagccgggataactccaaaaacacggtctatctccagatgaacagcctcagggccgaggacacagctacgtattactgtgcctttgaggagcactggatgacacctctgtactggggacaggggacgcaggtaactgtgagtagc

SEQ ID NO: 34 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggacgtacatttcgtatcgacacaatgggctggtttcgtcaggcccctggcaaggagagagagttcgtttccgccatctcgtttgacacaagtcatgagaactattacgctgacagcgtaaagggaagatttacaattagccgggataactccaaaaacacggtctatctccagatgaacagcctcagggccgaggacacagctacgtattactgtgcccctgagtcgatgtggttttcgattgagtattggggacaggggacgcaggtaactgtgagtagc

SEQ ID NO: 35 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggcacaactagttttcgtgacacaatgggctggtttcgtcaggcccctggcaaggagagagagttcgtttccgccatctcgtggaacgaagggcatgcacattactacgctgacagcgtaaagggaagatttacaattagccgggataactccaaaaacacggtctatctccagatgaacagcctcagggccgaggacacagctacgtattactgtgcctcggaggacatgtggtttaaccacttttattggggacaggggacgcaggtaactgtgagtagc

SEQ ID NO: 36 - DNA, 372 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggatcaactagtagtcattacgacatgggctggtttcgtcaggcccctggcaaggagagagagttcgtttccgccatctcgtacaaccatgaccatgacacatactacgctgacagcgtaaagggaagatttacaattagccgggataactccaaaaacacggtctatctccagatgaacagcctcagggccgaggacacagctacgtattactgtgccggtgcaacagcaacaacagacggtggttatcaggagtattggggacaggggacgcaggtaactgtgagtagc

SEQ ID NO: 37 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaacatcctacgcaaacagtgacatgggctggtttcgtcaggcccctggcaaggagagagagttcgtttccgccatctcgggtggtgacagtgcatattactattacgctgacagcgtaaagggaagatttacaattagccgggataactccaaaaacacggtctatctccagatgaacagcctcagggccgaggacacagctacgtattactgtgccacacagcagattggtatgcgtgagtattattggggacaggggacgcaggtaactgtgagtagc

SEQ ID NO: 38 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggatcatacagtgcagagacagacatgggctggtttcgtcaggcccctggcaaggagagagagttcgtttccgccatctcgggtgaccctacaacaacaacatactacgctgacagcgtaaagggaagatttacaattagccgggataactccaaaaacacggtctatctccagatgaacagcctcagggccgaggacacagctacgtattactgtgcatatcagcgtctggacaactttgagatttattggggacaggggacgcaggtaactgtgagtagc

SEQ ID NO: 39 - DNA, 372 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggcttcttctcaaacatcgaggacatgggctggtttcgtcaggcccctggcaaggagagagagttcgtttccgccatctcgggttatgaaaacgagtcgccttattacgctgacagcgtaaagggaagatttacaattagccgggataactccaaaaacacggtctatctccagatgaacagcctcagggccgaggacacagctacgtattactgtgcagcacagtcgtatcacggtaagaagggtatgacagagtattggggacaggggacgcaggtaactgtgagtagc

SEQ ID NO: 40 - DNA, 372 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggacgtacatcatggtcggaaattatgggctggtttcgtcaggcccctggcaaggagagagagttcgtttccgccatctcgcgttatacagacgactattcgtactacgctgacagcgtaaagggaagatttacaattagccgggataactccaaaaacacggtctatctccagatgaacagcctcagggccgaggacacagctacgtattactgtgcaacatatcactatcagtatggtggtgaccagacattttactggggacaggggacgcaggtaactgtgagtagc

SEQ ID NO: 41 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagcatacagtgcacgtacaacaatgggctggtttcgtcaggcccctggcaaggagagagagttcgtttccgccatctccgcacatgaagggcaggcaccttattacgctgacagcgtaaagggaagatttacaattagccgggataactccaaaaacacggtctatctccagatgaacagcctcagggccgaggacacagctacgtattactgtgccacacgtgcacacgagcctaagggtatgtactggggacaggggacgcaggtaactgtgagtagc

SEQ ID NO: 42 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagcatcctatcgtggtacatctatgggctggtttcgtcaggcccctggcaaggagagagagttcgtttccgccatctcctttcgtcctgaccagcagccttactacgctgacagcgtaaagggaagatttacaattagccgggataactccaaaaacacggtctatctccagatgaacagcctcagggccgaggacacagctacgtattactgtgcagagcgtcgtcagcactcgcgtggtctgtactggggacaggggacgcaggtaactgtgagtagc

SEQ ID NO: 43 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggacgtacattcgacggttccgcaatgggctggtttcgtcaggcccctggcaaggagagagagttcgtttccgccatctcggcatcgccttcgggtgagtcgtattacgctgacagcgtaaagggaagatttacaattagccgggataactccaaaaacacggtctatctccagatgaacagcctcagggccgaggacacagctacgtattactgtgccggtcgtggtcgttcgcagaagtcgatgtattggggacaggggacgcaggtaactgtgagtagc

SEQ ID NO: 44 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagatacatggcgtacaactgcaatgggctggtttcgtcaggcccctggcaaggagagagagttcgtttccgccatctcgggtggtgacaaccgtggtacatattacgctgacagcgtaaagggaagatttacaattagccgggataactccaaaaacacggtctatctccagatgaacagcctcagggccgaggacacagctacgtattactgtgccggtcgtctgaaccagaacggtcctatgtactggggacaggggacgcaggtaactgtgagtagc

SEQ ID NO: 45 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagatggatcatttgcatccgcaatgggctggtttcgtcaggcccctggcaaggagagagagttcgtttccgccatctcggcacatgcaggtggttcgccttattacgctgacagcgtaaagggaagatttacaattagccgggataactccaaaaacacggtctatctccagatgaacagcctcagggccgaggacacagctacgtattactgtgcacagcgtattggtgcaacacgtgcaatgtactggggacaggggacgcaggtaactgtgagtagc

SEQ ID NO: 46 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggcttcacaagtggttcgacagcaatgggctggtttcgtcaggcccctggcaaggagagagagttcgtttccgccatctcgcgttttgaaaaccagggtaactattacgctgacagcgtaaagggaagatttacaattagccgggataactccaaaaacacggtctatctccagatgaacagcctcagggccgaggacacagctacgtattactgtgcaacacgtgcagttgcatcgtcgacaatgtactggggacaggggacgcaggtaactgtgagtagc

SEQ ID NO: 47 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaggcttttttacatcgagtacaatgggctggtttcgtcaggcccctggcaaggagagagagttcgtttccgccatctcccgtcaacatgacaacgcaccttattacgctgacagcgtaaagggaagatttacaattagccgggataactccaaaaacatggtctatctccagatgaacagcctcagggccgaggacacagctacgtattactgtgccgcacgtaacgcaacacacaagggtatttactggggacaggggacgcaggtaactgtgagtagc

SEQ ID NO: 48 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggcggcacatcatttcagacagcaatgggctggtttcgtcaggcccctggcaaggagagagagttcgtttccgccatctcgagtcgtcaaggttcgcatccttattacgctgacagcgtaaagggaagatttacaattagccgggataactccaaaaacacggtctatctccagatgaacagcctcagggccgaggacacagctacgtattactgtgcaacacgtatgacagagacacgtaagatgtactggggacaggggacgcaggtaactgtgagtagc

SEQ ID NO: 49 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggatcaacttttaagcattccgcaatgggctggtttcgtcaggcccctggcaaggagagagagttcgtttccgccatctcgagtggtcctggtatggagtcgtactatgctgacagcgtaaagggaagatttacaattagccgggataactccaaaaacacggtctatctccagatgaacagcctcagggccgaggacacagctacgtattactgtgcaggtcgtgcatttcctggtcagaagatgtactggggacaggggacgcaggtaactgtgagtagc

SEQ ID NO: 50 - DNA, 390 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaacaggctcaaacgcatccgcaatgggctggtttcgtcaggcccctggcaaggagagagagttcgtttccgccatctcccgtcatgaaggtcagcctccttactacgctgacagcgtaaagggaagatttacaattagccgggataactccaaaaacacggtctatctccagatgaacagcctcagggccgaggacacagctacgtattactgtgcaatgcgttatcagtcgcacattgacattcctggtgactcgtcgcctaagctgatgtattggggacaggggacgcaggtaactgtgagtagc

SEQ ID NO: 51 - DNA, 381 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggcgatactagtggtgcaacagtaatgggctggtttcgtcaggcccctggcaaggagagagagttcgtttccgccatctccagttgggcaagtatttggccttactacgctgacagcgtaaagggaagatttacaattagccgggataactccaaaaacacggtctatctccagatgaacagcctcagggccgaggacacagctacgtattactgtgcctataagctgtatggtgacatgacaatggagtggggttttaagtcgtattggggacaggggacgcaggtaactgtgagtagc

SEQ ID NO: 52 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagatactagtaaccgtgacgcaatgggctggtttcgtcaggcccctggcaaggagagagagttcgtttccgccatctcgggtcatggtagtacacagggttactacgctgacagcgtaaagggaagatttacaattagccgggataactccaaaaacacggtctatctccagatgaacagcctcagggccgaggacacagctacgtattactgtgccgagctggaccgtgagtggggtaagtggtactggggacaggggacgcaggtaactgtgagtagc

SEQ ID NO: 53 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggatcaacatacggtcaggatacaatgggctggtttcgtcaggcccctggcaaggagagagagttcgtttccgccatctcgagttattcgtcggttgacggttactacgctgacagcgtaaagggaagatttacaattagccgggataactccaaaaacacggtctatctccagatgaacagcctcagggccgaggacacagctacgtattactgtgcaattattgacgagcagtttcacatgccttattggggacaggggacgcaggtaactgtgagtagc

SEQ ID NO: 54 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggcacatactcagaggagactgcaatgggctggtttcgtcaggcccctggcaaggagagagagttcgtttccgccatctcgtttgcacaagacggttatcgttactacgctgacagcgtaaagggaagatttacaattagccgggataactccaaaaacacggtctatctccagatgaacagcctcagggccgaggacacagctacgtattactgtgcagagtttgacaacgtttggcacctgccttattggggacaggggacgcaggtaactgtgagtagc

SEQ ID NO: 55 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggatcaacaagtgcagaggagtctatgggctggtttcgtcaggcccctggcaaggagagagagttcgtttccgccatctcggcacaacaaggtgcagagccttactacgctgacagcgtaaagggaagatttacaattagccgggataactccaaaaacacggtctatctccagatgaacagcctcagggccgaggacacagctacgtattactgtgcagagtttgacaacgtttggcacctgccttattggggacaggggacgcaggtaactgtgagtagc

SEQ ID NO: 56 - DNA, 372 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaacaacatcatggcgtgagacaatgggctggtttcgtcaggcccctggcaaggagagagagttcgtttccgccatctcgtacggttgggactcggttggttattacgctgacagcgtaaagggaagatttacaattagccgggataactccaaaaacacggtctatctccagatgaacagcctcagggccgaggacacagctacgtattactgtgccattgagacatatggtgcatcgaacggttggcgtgagtactggggacaggggacgcaggtaactgtgagtagc

SEQ ID NO: 57 - DNA, 381 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaggctatttcacaatcgaggacatgggctggtttcgtcaggcccctggcaaggagagagagttcgtttccgccatctccttttggtggacgaacacacattactacgctgacagcgtaaagggaagatttacaattagccgggataactccaaaaacacggtctatctccagatgaacagcctcagggccgaggacacagctacgtattactgtgcccctatgacagagcgtgcagaggcacagatgggtacaatggagccttactggggacaggggacgcaggtaactgtgagtagc

SEQ ID NO: 58 - DNA, 363 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggaacatatagtaaggagactgacatgggctggtttcgtcaggcccctggcaaggagagagagttcgtttccgccatctccgactggcctgactattatggttactacgctgacagcgtaaagggaagatttacaattagccgggataactccaaaaacacggtctatctccagatgaacagcctcagggccgaggacacagctacgtattactgtgcatgggttaacgagaagtttggttatgtttattggggacaggggacgcaggtaactgtgagtagc

SEQ ID NO: 59 - DNA, 372 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggagcaacttcaaagtcgtacggtatgggctggtttcgtcaggcccctggcaaggagagagagttcgtttccgccatctcgagttggaacaacgactggtcgtactacgctgacagcgtaaagggaagatttacaattagccgggataactccaaaaacacggtctatctccagatgaacagcctcagggccgaggacacagctacgtattactgtgcatatgacctgcagcacggttcgggtatgcctcgtatgtattggggacaggggacgcaggtaactgtgagtagc

SEQ ID NO: 60 - DNA, 390 bp, Artificial Sequence
  Derived from a cartilaginous fish heavy chain antibody or a camelid heavy chain nanobody
atggcggaagtgcagctgcaggcttccgggggaggatttgtgcagccgggggggtcattgcgactgagctgcgccgcatccggctcaacatcagagatcacaggtatgggctggtttcgtcaggcccctggcaaggagagagagttcgtttccgccatctcggaatggcctggtaactatgcatattacgctgacagcgtaaagggaagatttacaattagccgggataactccaaaaacacggtctatctccagatgaacagcctcagggccgaggacacagctacgtattactgtgcaggtgacggtctggcatcggacttttatggtaagattgacgagctggacatgatgtactggggacaggggacgcaggtaactgtgagtagc